(19) United States
(12) Patent Application Publication
(10) Pub. No.: US 2024/0285553 A1
(43) Pub. Date: Aug. 29, 2024

(54) METHODS TO MODULATE MITOPHAGY WITH SREBP SIGNALING PATHWAY ACTIVATORS

(71) Applicant: THE TRUSTEES OF INDIANA UNIVERSITY, Bloomington, IN (US)

(72) Inventors: Ankit A. DESAI, Carmel, IN (US); Samisubbu R. NAIDU, Zionsville, IN (US)

(21) Appl. No.: 18/572,621

(22) PCT Filed: Jul. 6, 2022
(86) PCT No.: PCT/US2022/073445

§ 371 Date: Dec. 20, 2023

Related U.S. Application Data
(60) Provisional application No. 63/219,109, filed on Jul. 7, 2021.
(60) Provisional application No. 63/232,677, filed on Aug. 13, 2021.

(51) Int. Cl. A61K 31/138; A61K 31/165; A61K 31/566; A61P 9/10

(52) U.S. Cl. CPC A61K 31/138; A61K 31/165; A61K 31/566; A61P 9/10

(57) ABSTRACT

Disclosed are methods to stimulate intracellular mitophagy in a cell nucleus of a subject in need thereof, by administering to the subject an effective amount of a sterol regulatory element-binding protein (SREBP) signaling pathway activator that induces nuclear translocation of SREBP C-terminal regulatory domain into the cell nucleus.

### CROSS REFERENCE TO RELATED APPLICATIONS

This application claims priority to the following: U.S. Provisional Patent Application No. 63/219,109 filed on Jul. 7, 2021 and U.S. Provisional Patent Application No. 63/232,677 filed on Aug. 13, 2021, the disclosure of which are expressly incorporated herein.

### FIELD

The present disclosure relates to the field of modulating mitophagy. More specifically, the present disclosure relates to methods to modulate mitophagy with a sterol regulatory element-binding protein (SREBP) signaling pathway activator.

### BACKGROUND

To maintain healthy mitochondria, a constant need exists to generate new mitochondria components while removing the damaged ones. The proper functioning of this intracellular “quality-control” mechanism is fundamental to both mitochondrial and cellular homeostasis. Mitophagy, a selective degradation of mitochondria (typically, damaged or malfunctioning) by autophagy (usually via lysosomes), is a vital mechanism to maintain mitochondrial quality control in cells. Mitochondrial autophagy is the only known pathway that mediates the turnover of whole mitochondria to avoid cellular damage and apoptosis.

Based on prior literature, lipid and fatty acid accumulation and oxidation is associated with mitochondrial injury. Briefly, lipids and cholesterol are essential for normal physiological processes such as maintaining integrity of cell membrane and membranes of internal organelles, providing a source of energy, cell signaling to regulate a host of functions such as cell proliferation, metabolism, inflammation and apoptosis. Disruption of lipid homeostasis can promote human disease.

Lipid oxidation usually occurs in the mitochondria while the cytosol is the site of lipid synthesis. Oxidized cell membrane lipids are often pathological and any defects in replacement of oxidized membrane lipid with newly made lipid can further exacerbate pathological conditions. Yet, how lipid synthesis is coordinated with mitochondrial clearance remains unclear. Despite observations of impairment of mitophagy in many disease settings, there is no clinical candidate or drug used in the clinical setting reported to robustly induce mitophagy to treat diseases.

As disclosed herein, applicant has discovered that propranolol, a drug that is used in various clinical settings, promotes mitophagy and stimulates lipid synthesis via a molecule called SREBP (sterol regulatory element-binding protein). The ability to promote mitophagy through the use of activators of SREBP has the potential to impact many diseases.

### SUMMARY

Mitophagy is an important cellular process involved in clearing degraded/damaged/dead mitochondria from a cell. Reduced or impaired mitophagy plays a role in the initiation or exacerbation of a spectrum of human diseases (Parkinson's, Alzheimer's, cardiovascular, cancer, aging-related diseases). Accordingly, counteracting defective or inadequate cellular mitophagy is anticipated to be an effective strategy to treat disease states associated with defective or inadequate cellular mitophagy. As disclosed herein, applicant has discovered that propranolol (S and R), NCEH1 inhibitors, and SREBP1 agonists can robustly induce mitophagy via target signaling cascade. More particularly, NCEH1 has been determined herein to directly and specifically binds to both phosphatidylserine (PS) and SREBP1, retaining SREBP1 in the cytosol. Applicant has found that disruption of the NCEH1-PS interaction leads to a noncanonical activation of SREBP1 (See FIG. 7A). Applicant further observed that use of JW480, a selective NCEH1 inhibitor, protects mice in two in vivo mouse models of CV injury: induction of large myocardial infarction (MI) & laser-induced choroidal neovascularization (CNV)).

Via 2 specific aims (SAs), we propose to test the hypothesis that propranolol or JW480 disrupts NCEH1-PS interactions and promotes non-canonical activation of SREBP1.

In one aspect, the present disclosure relates to a method to stimulate intracellular mitophagy in a cell of a subject in need thereof by administering to the subject a sterol regulatory element-binding protein (SREBP) signaling pathway activator in an amount effective to stimulate the cellular mitophagy in the subject. Activation of SREBP induces nuclear translocation of SREBP C-terminal regulatory domain into the cell nucleus and stimulates new lipid generation for optimal cell membrane lipid composition, and promote cell survival, in part, by repairing membrane damage and inducing mitophagy.

In another aspect, the disclosure relates to a method to stimulate intracellular lipid biosynthesis in a cell nucleus of a subject in need thereof by administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the intracellular lipid synthesis in the subject.

In another aspect, the disclosure relates to a method to treat an intracellular mitophagy dysfunction-associated disease in a subject in need thereof by administering to the subject an effective amount of a sterol regulatory element-binding protein (SREBP) signaling pathway activator that induces nuclear translocation of SREBP C-terminal regulatory domain into a cell nucleus.

In another aspect, the disclosure relates to a method to stimulate intracellular mitophagy in a cell of a subject in need thereof by administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into the cell nucleus in an amount sufficient to stimulate cellular mitophagy in the subject.

In another aspect, the disclosure relates to a method to stimulate intracellular lipid biosynthesis in a cell nucleus of a subject in need thereof, the method comprising the step of: administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into the cell nucleus in an amount sufficient to stimulate the intracellular lipid synthesis in the subject.

In another aspect, the disclosure relates to a method to treat an intracellular mitophagy dysfunction-associated disease in a subject in need thereof by administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into a cell nucleus.

In one embodiment a pharmaceutical composition is provided comprising a SREBP signaling pathway activator and a pharmaceutically acceptable carrier, wherein said activator is selected from the group consisting of propranolol, R-propranolol, U18666A, JW480 and pharmaceutically acceptable salts of propranolol, R-propranolol, U18666A, and JW480.

In another aspect, the disclosure relates to a method to identify a biomarker for cellular mitophagy in a target tissue of a mammal by: (a) administering a sterol regulatory element-binding protein (SREBP) signaling pathway activator that induces SREBP C-terminal regulatory domain translocation into the cell nucleus; (b) collecting endothelial cells from a blood sample of the mammal; (c) analyzing the collected mammalian endothelial cells for cellular mitophagy; (d) collecting cells from the target tissue of the mammal; (e) analyzing the collected mammalian target tissue cells for cellular mitophagy; and, (f) determining a correlation between cellular mitophagy in the mammalian endothelial cells and cellular mitophagy in the mammalian target tissue cells.

In another aspect, the disclosure relates to a method of identifying a candidate sterol regulatory element-binding protein (SREBP) signaling pathway activator by: (a) contacting a cell with the candidate SREBP signaling pathway activator; and, (b) analyzing the contacted cell for mitophagy and for presence of SREBP C-terminal regulatory domain or for presence of SREBP N-terminal transactivation domain and neutral cholesterol ester hydrolase 1 (NCEH1) translocation in the cell nucleus.

In another aspect, the disclosure relates to a method to stimulate intracellular mitophagy in a cell nucleus of a subject in need thereof by administering to the subject an effective amount of propranolol, JW480, or a pharmaceutically acceptable salts thereof sufficient to stimulate the cellular mitophagy in the subject.

In another aspect, the disclosure relates to a method to stimulate intracellular lipid biosynthesis in a cell nucleus of a subject in need thereof by administering to the subject an effective amount of propranolol, JW480, or a pharmaceutically acceptable salts thereof sufficient to stimulate the intracellular lipid synthesis in the subject.

In another aspect, the disclosure relates to a method to treat an intracellular mitophagy dysfunction-associated disease in a subject in need thereof by administering to the subject an effective amount of propranolol, JW480, or a pharmaceutically acceptable salts thereof.

Additional embodiments, features, and advantages of the disclosure will be apparent from the following detailed description and through practice of the disclosure.

### BRIEF DESCRIPTION OF THE DRAWINGS

FIGS. 1A-1C illustrate propranolol promotes mitophagy in pulmonary arterial endothelial cells (PAECs). The TIM/TOM complex is a protein complex in cellular biochemistry which translocates proteins produced from nuclear DNA through the mitochondrial membrane for use in oxidative phosphorylation. TOM20 is a peripheral component of the TOM complex that functions as a primary receptor for mitochondrial precursor proteins and serves as a mitochondrial marker. FIG. 1A: Donor- or pulmonary arterial hypertension (PAH) patient-derived PAEC were treated with carbonyl cyanide m-chlorophenylhydrazone (CCCP) and blotted with TOM20 antibodies showing reduced TOM20 with CCCP in control cells, consistent with its known role in the induction of mitophagy, but demonstrated lack of mitophagy induction in diseased cells with mixed TOM20 expression. FIG. 1B: PAH (diseased)-derived PAEC were treated with vehicle or propranolol and blotted with TOM20 and showed reduction in TOM20, suggesting ability of propranolol to induce mitophagy in diseased cells. FIG. 1C provides a quantification of TOM20 levels from Panel A. ***: p<0.001 PAECs treated with propranolol and stained with antibodies showed a reduced TOM20/Lamp1 co-staining with propranolol, providing microscopic immunofluorescence evidence of mitophagy induction.

FIG. 2 illustrates that propranolol promotes mitophagy in patient cells. Normal control endothelial cells (ECs) or patient-derived ECs were treated with propranolol and Tom20 levels were assessed showing reduced TOM20 across all cells, suggesting robust ability to induce mitophagy under control and diseased conditions.

FIG. 3 illustrates that propranolol induces mitophagy by TEM analysis in ECs. Transmission electron microscopy shows evidence of significant mitophagy in ECs from PAH patients after exposure to propranolol but not by vehicle.

FIG. 4 illustrate that propranolol attenuates MCT (monocrotaline)-induced PAH in rats. More particularly, FIG. 4 is a graph presenting right ventricular systolic pressures (RVSP) measurements of rats exposed to MCT and treated with vehicle or propranolol for the last 2 of a 4-week PAH rodent model; ***: p<0.001. MCT injection induces severe PAH and is a well-accepted PAH model in rats. Propranolol reduces RVSP or right ventricular systolic pressures which is a surrogate of PAH presence and severity. Rat lung sections were stained with H&E and the histology analysis showed vessels were much less remodeled and vessel thickness is attenuated after exposure to propranolol, further supporting propranolol's ability to attenuate PAH lung vascular remodeling and disease pathogenesis.

FIG. 5 illustrates that R-propranolol promotes mitophagy in PAECs. ECs exposed to propranolol or R-propranolol (which has 100-fold less beta-adrenergic receptor affinity) and Tom20 levels assessed. These data show that both propranolol and R-propranolol reduce TOM20, suggesting they both induce mitophagy in ECs and that this process likely is beta-adrenergic receptor independent.

FIGS. 6A-6B illustrate that propranolol activates SREBP pathway. PAEC treated with propranolol in the presence or absence of fatostatin, a known SREBP1 inhibitor, and blotted as indicated. As shown in FIG. 6A, TOM20 levels go down with propranolol, suggesting mitophagy and SREBP1 levels go down with propranolol, suggesting SREBP1 cleavage and activation. SCD, which is a target of SREBP1, is increased with propranolol, suggesting SREBP1 activation. Fatostatin inhibits SREBP1 activation resulting in increased non-cleaved SREBP1 and reduced SCD and increased TOM20 (all contrasting results to propranolol). Propranolol is able to rescue inhibitor effects of fatostatin as shown in the last lane of the western blot. FIG. 6B. Other beta-blockers do not induce mitophagy as shown by increases in TOM20 in EC lysates after exposure to these other beta-blockers.

FIGS. 7A & 7B: FIG. 7A is a schematic drawing showing an alternate pathway of SREBP1 activation by NCEH1 inhibition. FIG. 7B illustrates that neutral cholesterol ester hydrolase 1 (NCEH1) inhibition activates SREBP1 and promotes mitophagy. Left panel shows the structures of indicated compounds. Right panel shows western blot of cells treated with indicated drugs and blotted with the antibodies shown. Propranolol, R-propranolol, and JW480 all reduce SREBP1 levels, indicating SREBP1 cleavage and activation. SCD is elevated with all three drugs, indicating all three can activate SREBP1 activation. NCEH1 levels are also higher in all three conditions, indicating there may be a compensatory rise in total NCEH1 levels with NCEH1 inhibition. TOM20 is lower with all of the drugs, indicating they all induce mitophagy.

FIG. 8 illustrates that NCEH1 binds to SREBP1. ECs were exposed to vehicle or propranolol and SREBP1 protein complex was captured on anti-SREBP1 antibody. A Western Blot revealed that NCEH1 associates/binds with SREBP1.

FIGS. 9A-9B-NCEH1 interacts with SREBP1 at the transactivation domain. Either SREBP1 C-terminal regulatory domain mutant or SREBP1 full length proteins (both HA tagged) were transfected into human cells (HEK293), along with Flag tagged NCEH1 (See FIG. 9A providing a schematic drawing of the epitope tagged NCEH1/SREBP1 fragments for binding assay. As shown in FIG. 9B, NCEH1 interacts with both N- and C-termini of SREBP1 as both SREBP1 proteins were pulled down from cell lysate using HA antibody beads and blotted with Flag antibodies to detect NCEH1 interaction with SREBP1 proteins. These data show NCEH1 potentially interacts with transactivation domain of SREBP1.

FIGS. 10A-10C-R-propranolol attenuates rodent PAH. Sugen (20 mg/kg, IP) injection was administered to rats and then they were exposed to 3 weeks of hypoxia (10% FiO2) and then placed in normoxia (21% FiO2) for 4 weeks to develop severe pulmonary arterial hypertension (FIG. 10A is a schematic of the experimental procedure). Rats were then administered either r-propranolol (5 mg/kg/day, IP) for the last 4 weeks or r-propranolol (10 mg/kg/day, IP) for the last 2 weeks or vehicle for the last 4 weeks. Rats with vehicle exposure developed severe PAH at the end of the protocol as confirmed by right ventricular systolic pressures (RVSP; FIG. 10B) and surrogate measures of RV hypertrophy (RV/(LV+S); FIG. 10C). R-propranolol exposure attenuated both RVSP and RV hypertrophy at the higher dose (10 mg/kg/day) and trended toward improvement with the lower dose (5 mg/kg/day).

FIGS. 11A-11E: FIG. 11A show a schematic of the dot blot membrane strip used to demonstrate that NCEH1 specifically binds to phosphatidylserine (PS) in vitro. Membranes having the specified compounds were incubated with purified NCEH1 protein and the membrane was developed with NCEH1 antibody. FIG. 11B shows NCEH1 specifically binds to phosphatidylserine (PS) in a dose dependent manner. FIG. 11C show a schematic of the dot blot membrane strip used to demonstrate that that propranolol/JW480 disrupts NCEH1-phosphatidylserine interactions in vitro. Membranes having the specified compounds were incubated with purified NCEH1 protein in the presence of vehicle, Propranolol or JW480 and the membrane was the developed with NCEH1 antibody. FIG. 11D provides data showing that Propranolol/JW480 disrupts NCEH1-phosphatidylserine interactions. FIG. 11E demonstrates that a known phosphatidylserine binding drug (papuamide) recapitulates JW480 activity. ECs were treated with papuamide and cell extracts were blotted with indicated antibodies. Papuamide was found to activate a non-canonical SREBP1 pathway similar to NCEH1 inhibitor JW480 and papuamide promotes nuclear translocation of SREBP1 in ECs.

FIG. 12: NCEH1 inhibition provides protection from heart failure in myocardial infarction (MI) mouse model.

FIGS. 13A & 13B: NCEH1 inhibition protects from vascular leakage in mouse CNV model. FIG. 13A shows average leakage per eye with or without JW480 administration and FIG. 13B shows the average of CNV per eye with or without JW480 administration.

FIG. 14: Using a mouse model fed a high-fat diet, AKR/J mice develop obesity, insulin resistance, heart failure with preserved ejection fraction, and pulmonary hypertension and are designated as “HF-PH mice”. These mice were administered propranolol or R-propranolol and FIG. 14 provides the right ventricular systolic pressure of HF-PH mice after 4 weeks of treatment (N=5-7).

FIGS. 15A-15M-R-propranolol attenuates rodent PAH. Sugen (20 mg/kg, IP) injection was administered to rats and then they were exposed to 3 weeks of hypoxia (10% FiO2) and then placed in normoxia (21% FiO2) for 4 weeks to develop severe pulmonary arterial hypertension (FIG. 15A is a schematic of the experimental procedure). Rats receiving Sugen (SuHx) for three weeks of 7 weeks were then administered either r-propranolol (5 mg/kg/day, IP) for the last 4 weeks or r-propranolol (10 mg/kg/day, IP) for the last 2 weeks or vehicle for the last 4 weeks. SuHx treated rats (but not controls) with vehicle exposure developed severe PAH at the end of the protocol as confirmed by right ventricular pressures (RVP; FIG. 15B) and right ventricular systolic pressures (RVSP; FIG. 15C, lane 1: control; lane 2: control+r-propranolol 5 mg; lane 3: SuHx 3 wk; lane 4: SuHx 7 wk; lane 5: SuHx+r-propranolol 5 mg; SuHx+r-propranolol 10 mg) and surrogate measures of RV hypertrophy (FIGS. 15D-15J). R-propranolol exposure attenuated both RVSP and RV hypertrophy at the higher dose (10 mg/kg/day) and trended toward improvement with the lower dose (5 mg/kg/day). FIG. 15K show the PA thickness of SuHx treated rats with vehicle exposure vs r-propranolol exposure. FIG. 15L show the RV cross-sectional thickness of SuHx treated rats with vehicle exposure vs r-propranolol exposure.

FIG. 15M show the interstitial fibrosis of SuHx treated rats with vehicle exposure vs r-propranolol exposure.

### DETAILED DESCRIPTION

The present disclosure is directed to the discovery, first disclosed herein, of a non-canonical SREBP signaling pathway activation that stimulates mitophagy.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to are incorporated by reference in their entireties. If a definition set forth in this section is contrary to or otherwise inconsistent with a definition set forth in a patent, application, or other publication that is incorporated by reference, the definition set forth in this section prevails over the definition incorporated by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the methods represented by the variables are specifically embraced by the present disclosure and are disclosed just as if each and every combination was individually and explicitly disclosed. In addition, all subcombinations of the features listed in the embodiments describing such variables are also specifically embraced by the present disclosure and are disclosed just as if each and every such sub-combination of chemical groups was individually and explicitly disclosed.

### Definitions

“Activator” refers to any substance that induces a conformational change or other alteration in a target compound. For example, an activator of the SREBP signaling pathway (which is known to regulate lipid synthesis) includes and activator that activates or increases the cellular signal transduction of SERBP-mediated signals. Signal transduction refers to the transmission of a molecular signal in the form of chemical modification by recruitment of protein complexes along a pathway that ultimately triggers a biochemical event in the cell. An activator may be an “inhibitor.”

Cell” refers to the basic structural and functional unit of a living organism. In higher organisms, e.g., animals, cells having similar structure and function generally aggregate into “tissues” that perform particular functions. Thus, a tissue includes a collection of similar cells and surrounding intercellular substances, e.g., epithelial tissue, connective tissue, muscle, nerve. An “organ” is a fully differentiated structural and functional unit in a higher organism that may be composed of different types of tissues and is specialized for some particular function, e.g., kidney, heart, brain, liver, etc. Accordingly, by “specific organ, tissue, or cell” is meant herein to include any particular organ, and to include the cells and tissues found in that organ.

A “Biomarker” is a detectable signal or compound that directly or indirectly correlates to the presence of a metabolite, condition or disease. In accordance with one embodiment biomarkers are used to detect or measure the level of mitophagy in the nucleus of a cell or the activity or severity of a disease process. By way of a non-limiting example, mitophagy can be quantified by the expression of mitochondrial proteins using immunoblot assays or immunostaining, by mitochondria DNA and by microscopy. Exemplary genes that encode mitochondrial proteins include TOM20, LC3 and/or P62. In one embodiment a “biomarker” is used to measure the level and/or activity of neutral cholesterol ester hydrolase 1 (NCEH1). Biomarkers include surrogate biomarkers, i.e., lab measurements or physical signs that substitute for a clinically meaningful endpoint that is a direct measure of how a patient feels, functions or survives and can predict the effect of a therapy. The level of a biomarker corresponds to a decrease in a severity of a disease if the level of the biomarker from the subject differs by a significant amount, preferably at least 10%, and more preferably 25%, 50%, 75%, or 100%.

“Co-administration” refers to administration of unit dosages of two or more bioactive agents, wherein the active agents are administered simultaneously, or sequentially within a timeframe where the first administered agent is still therapeutically active when the last co-administered agent is administered. In accordance with the present disclosure, the SREBP signaling pathway activators before or after administration of unit dosages of one or more additional therapeutic agents, for example, administration of the SREBP signaling pathway activators within seconds, minutes, or hours of the administration of one or more additional therapeutic agents. For example, in some embodiments, a unit dose of a SREBP signaling pathway activator is administered first, followed within seconds or minutes by administration of a unit dose of one or more additional therapeutic agents. Alternatively, in other embodiments, a unit dose of one or more additional therapeutic agents is administered first, followed by administration of a unit dose of a SREBP signaling pathway activator within seconds or minutes. In some embodiments, a unit dose of a SREBP signaling pathway activator is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a SREBP signaling pathway activator. Co-administration of a SREBP signaling pathway activator with one or more additional therapeutic agents generally refers to simultaneous or sequential administration of a SREBP signaling pathway activator and one or more additional therapeutic agents, such that therapeutically effective amounts of each agent are present in the body of the patient.

“Disease” means a state of health in which the subject cannot maintain homeostasis, and where if the disease is not ameliorated then the subject's health continues to deteriorate, or a state of health in which the subject is able to maintain homeostasis, but in which the subject's state of health is less favorable than it would be in the absence of the disease but does not necessarily cause a further deterioration in the subject's health.

“Effective amount” is that amount sufficient, at dosages and for periods of time necessary, to achieve a desired therapeutic result, such as for treatment of a disease (e.g. cancer), condition, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject. One skilled in the art will recognize that the condition of the individual can be monitored throughout the course of therapy and that the effective amount of a SREBP signaling pathway activator or composition containing it that is administered can be adjusted accordingly.

“Inhibitor” means any molecule that measurably reduces or blocks the activity of the target protein or molecule (i.e. the NCEH1 enzyme) or that measurably reduces expression of the gene product.

“Mitophagy” refers to selective degradation of mitochondria in the cell through autophagy, i.e., typically damaged mitochondria are engulfed by autophagosomes that then fuse with lysosomes which contain hydrolytic enzymes to break down the engulfed, damaged mitochondria. “Autophagy” is an evolutionarily conserved pathway involving the engulfment of cytosolic contents by a lipid membrane for recycling of organelles. Mitophagy is one form of macroautophagy that involves selectively targeting and engulfing mitochondria for removal through lysosomal degradation.

“Mitophagy dysfunction” means the cellular process of removing and/or degrading mitochondria is biologically ineffective, impaired, reduced or insufficient for the purposes of maintaining a healthy viable cell and proper cell function.

“Mitophagy dysfunction-associated disease” refers to a disease mediated or influenced, at least in part, by reduced or impaired mitophagy in mammalian cells.

“Neutral cholesterol ester hydrolase 1” refers to the human enzyme neutral cholesterol ester hydrolase 1 (NCEH1) and its mammalian homologs that hydrolyze 2-acetyl monoalkylglycerol ether to monoalkylglycerol ether and that is encoded by the NCEH1 gene. NCEH1 enzyme inhibitors embrace any compound, having the ability to block, partially or completely, the enzymatic hydrolysis of 2-acetyl monoalkylglycerol ether to monoalkylglycerol ether. In vivo, NCEH1 hydrolyzes cholesterol esters and release free cholesterol and lipids.

NCEH1 enzyme inhibitor also includes inhibitors of NCEH1 gene expression such as microRNA, shRNA, siRNA, antisense, or ribozyme molecules specifically targeted to a nucleic acid molecule encoding NCEH1 (e.g., human NCEH1 mRNA sequence and its mammalian homologs). Small molecule NCEH1 enzyme inhibitors include, but are not limited to propranolol, R-propranolol, and JW480.

“Non-regenerative” cells and tissue include cells and tissues that do not spontaneously regenerate (i.e, replicate or produce new daughter cells from old parent cells) such as neurons (central and peripheral nervous system), cardiomyocytes (heart muscle cells), skeletal-muscle cells, insulin-producing cells (beta-cells of the endocrine pancreas), and retinal pigment epithelium.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to pharmaceutically acceptable organic or inorganic salts of a SREBP signaling pathway activator. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound.

Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. Lists of other suitable pharmaceutically acceptable salts are found in Remington: The Science and Practice of Pharmacy, 21^{st} Edition, Lippincott Wiliams and Wilkins, Philadelphia, Pa., 2006.

“Safe and effective amount” means an amount of the SREBP signaling pathway activator sufficient to treat a condition but low enough to avoid serious side effects (at a reasonable benefit/risk ratio) within the scope of sound medical judgment. A safe and effective amount of a SREBP signaling pathway activator will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

“Selective” inhibitor refers to the propensity of an inhibitor to bind to and/or inhibit the activity of one specific protein or to downregulate its expression in preference to other proteins. “Selective” pathway activator refers to the propensity of an activator to activate one specific pathway in preference to other pathways. A selective NCEH1 inhibitor has a greater preference for binding to NCEH1 or downregulating its expression over other serine hydrolase enzymes; a non-selective NCEH1 inhibitor not only inhibit NCEH1 but also related enzymes. A selective SREBP1 signaling pathway activator has a greater preference for activating the SREBP1 signaling pathway activator over other lipid synthesis pathways; a non-selective SREBP1 pathway activator activates both SREBP1 and related proteins.

“Small molecule” refers to a low molecular weight organic compound. A compound with a molecular weight of about 2000 daltons or less, preferably of about 500 to about 900 daltons or less is a small molecule. Small molecules may be natural or artificial. Biopolymers such as nucleic acids and proteins, and polysaccharides (such as starch or cellulose) are not small molecules—though their constituent monomers, ribo- or deoxyribonucleotides, amino acids, and monosaccharides, respectively, are often considered small molecules. Small molecules include pharmaceutically acceptable salts of small molecules.

“Sterol regulatory element-binding protein (SREBP) signaling pathway activator” refers to any substance, selective or nonselective, that (1) enhances or promotes or activates SREBP signaling pathway activity, and/or (2) induces neutral cholesterol ester hydrolase 1 (NCEH1) translocation into the cell nucleus, and and/or (3) induces SREBP C-terminal regulatory domain and/or N-terminal domain and/or full/whole protein translocation into the cell nucleus. The translocated SREBP C-terminal regulatory domain may be linked to the SREBP N-terminal activation domain by a transmembrane segment and the translocated neutral cholesterol ester hydrolase 1 (NCEH1) may be bound to the SREBP C-terminal regulatory and/or N-terminal nuclear-bound domains. Exemplary SREBP signaling pathway activators include, but are not limited to, NCEH1 enzyme inhibitors. NCEH1 enzyme inhibitors embrace any substance, selective or nonselective, having the ability to block, partially or completely, the enzymatic hydrolysis of 2-acetyl monoalkylglycerol ether to monoalkylglycerol ether or hydrolysis of cholesterol ester into free cholesterol and lipids. NCEH1 enzyme inhibitor also includes inhibitors of NCEH1 gene expression such as microRNA, shRNA, siRNA, antisense, or ribozyme molecules specifically targeted to a nucleic acid molecule encoding NCEH1 (e.g., human NCEH1 mRNA sequence and its mammalian homologs). Nonselective SREBP signaling pathway activators include small molecule, nonselective NCEH1 inhibitors such as propranolol and R-propranolol. More selective SREBP signaling pathway activators include small molecule, selective NCEH1 inhibitors such as JW430.

“Stimulating” intracellular lipid synthesis means that the process of synthesizing lipids in a cell is stimulated thereby resulting in the production of new lipid molecules or modified lipids in the cell.

“Stimulating” intracellular mitophagy means that the mitochondrial degradation process in a cell is stimulated, thereby clearing mitochondria (typically, damaged or impaired mitochondria). This process can frequently result in an increase in fully functional mitochondria and a decrease in impaired mitochondria in conjunction with an increase in net intracellular mitochondrial function.

“Subject” refers to any mammal for whom diagnosis, treatment, or therapy is desired including mammals, e.g., humans, laboratory animals (e.g., primates, rats, mice, rabbits and guinea pigs), livestock (e.g., cows, sheep, goats, and pigs), household pets (e.g., dogs, cats, and rodents), and horses.

“Treat,” “treating” or “treatment” refer to an action to obtain a beneficial or desired clinical result including, but not limited to, alleviation or amelioration of one or more signs or symptoms of a disease (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a disease can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the disease. “Treat” covers any treatment in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic treatment may be administered before, during or after the onset of the disease. The inventive therapy may be administered during a symptomatic stage of the disease, and in some cases after the symptomatic stage of the disease.

### EMBODIMENTS

Reduced or impaired mitophagy can result in a spectrum of human diseases, including for example, Parkinson's, Alzheimer's, cardiovascular, cancer, and aging-related diseases. As disclosed herein, applicant has discovered that a sterol regulatory element-binding protein (SREBP) signaling pathway activators, such as propranolol (S and R), NCEH1 inhibitors, and SREBP1 agonists, can robustly induce mitophagy via a target signaling cascade. Accordingly, one aspect of the present disclosure is directed to methods of enhancing mitophagy in the cells of subjects who's cells exhibit a rate of mitophagy insufficient to maintain optimal cell function (i.e., “insufficient mitophagy”).

In one aspect, the present disclosure relates to a method of stimulating intracellular mitophagy in a cell of a subject in need thereof by administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the cellular mitophagy in the subject.

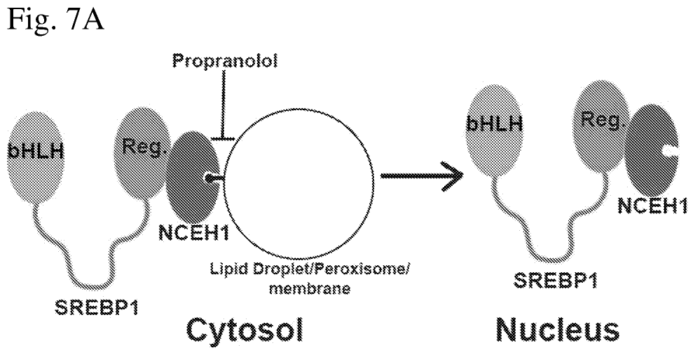

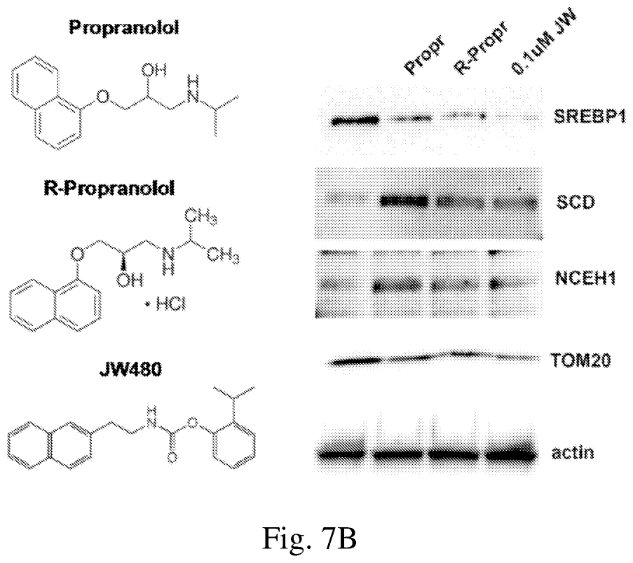

In one embodiment a method of enhancing mitophagy in cells exhibiting an insufficient capacity to process and remove damaged or defective mitochondria from the cell is provided. In one embodiment the cells are contacted in vivo in a subject that is experiencing detrimental effects of the cell's deficient capacity to remove damaged or defective mitochondria from the cell. In one embodiment the method comprises the step of contacting the cells with an activator of the sterol regulatory element-binding protein (SREBP) pathway. In one embodiment the activator of the SREBP pathway is a compound selected from the group consisting of S-propranolol, R-propranolol, U18666A and JW480. In one embodiment the activator of the SREBP pathway is JW480. In one embodiment the activator of the SREBP pathway is propranolol. In one embodiment two or more activators of the SREBP pathway are administered wherein the two or more activators are selected from S-propranolol, R-propranolol, U18666A and JW480. As used herein the term propranolol in the absence of any designated stereochemistry is intended to encompass both enantiomers. The structures of S-propranolol, R-propranolol and JW480 are provided in FIG. 7B. The structure of U18666A is

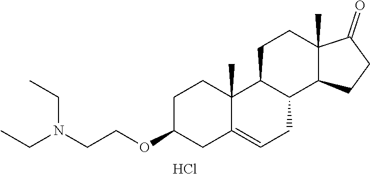 [SMILES: CCN(CC)CCO[C@H]1CC[C@@]2(C)C(=CC[C@@H]3[C@@H]2CC[C@]2(C)C(=O)CC[C@@H]32)C1 ; Cl]

In one embodiment the cells of a subject in need of enhanced mitophagy are contacted by administering a pharmaceutically acceptable composition comprising the activator of the SREBP pathway using any of the standard routes of administration, including for example by injection, optionally intravenous injection. In one embodiment the subject to be administered the mitophagy enhancing compositions of the present disclosure is a subject having a mitophagy dysfunction-associated disease. In one embodiment the subject has a mitophagy dysfunction-associated disease selected from the group consisting of mitochondrial disease, lung/respiratory disease, cardiovascular disease, liver disease, renal disease, neurodegenerative/neuropsychiatric disease and cancer.

In one embodiment the subject has a mitophagy dysfunction-associated disease selected from the group consisting of a lung/respiratory disease selected from the group consisting of bronchopulmonary dysplasia, chronic obstructive pulmonary disease, lung cancer, asthma, cystic fibrosis, pulmonary arterial hypertension, inflammatory lung disease, pleural cavity disease, pulmonary vascular disease, pneumonia, pulmonary embolism, idiopathic pulmonary fibrosis, sarcoidosis, mixed connective tissue disease, polymyositis, dermatomyositis, and systemic lupus erythematosus, optionally wherein the lung/respiratory disease is pulmonary arterial hypertension. In one embodiment the subject has a mitophagy dysfunction-associated disease selected from the group consisting of acute cardiac ischemic events, acute myocardial infarction, angina, arrhythmia, atherosclerosis, and chronic heart failure.

In accordance with one embodiment a method of increasing nuclear translocation of SREBP C-terminal regulatory domain into a cell nucleus is provided, wherein the method comprises the steps of contacting said cell with an activator of the sterol regulatory element-binding protein (SREBP) pathway. In one embodiment cells are contacted in vivo in a patient in need of increased mitophagy. In one embodiment the activator of the SREBP pathway is a compound selected from the group consisting of propranolol, U18666A and JW480. In one embodiment the activator of the SREBP pathway is JW480. In one embodiment the activator of the SREBP pathway is propranolol.

In accordance with one embodiment compositions are provided for inducing mitophagy in a cell. In one embodiment the pharmaceutical composition comprises a compound selected from the group consisting of propranolol, R-propranolol, U18666A and JW480 and a pharmaceutically acceptable carrier. In one embodiment the pharmaceutical composition comprises two or more compounds selected from the group consisting of R-propranolol, U18666A and JW480.

In one embodiment a method of treating mitophagy dysfunction is provided wherein the method comprises the step of contacting a cell exhibiting mitophagy dysfunction with a composition comprising an activator of the sterol regulatory element-binding protein (SREBP) pathway in an amount sufficient to increase nuclear translocation of SREBP C-terminal regulatory domain into the nucleus of said cell. In one embodiment the cell exhibiting mitophagy dysfunction is contacted with an activator of the SREBP pathway selected from the group consisting of propranolol, R-propranolol, U18666A and JW480.

In one embodiment a method of stimulating intracellular mitophagy in the cells of a subject in need thereof is provided. In one embodiment the method comprises the step of: administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the cellular mitophagy in the subject.

In one embodiment a method to stimulate intracellular lipid biosynthesis in the cells of a subject in need thereof is provided. In one embodiment, the method comprises the step of: administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the intracellular lipid synthesis in the subject. In one embodiment the activator of the SREBP pathway is selected from the group consisting of propranolol, R-propranolol, U18666A and JW480, optionally wherein the SREBP pathway activator is JW480.

In one embodiment a method to treat an intracellular mitophagy dysfunction-associated disease in a subject in need of treatment is provided, wherein the method comprises the step of: administering to the subject an effective amount of a sterol regulatory element-binding protein (SREBP) signaling pathway activator that induces nuclear translocation of SREBP C-terminal regulatory domain into a cell nucleus or a pharmaceutically acceptable salt thereof.

In one embodiment a method of stimulating intracellular mitophagy in a cell of a subject in need thereof is provided, wherein the method comprises the step of: administering to the subject an effective amount of a sterol regulatory element-binding protein (SREBP) signaling pathway activator (or pharmaceutically acceptable salt thereof) that induces nuclear translocation of SREBP N-terminal transactivation domain and neutral cholesterol ester hydrolase 1 (NCEH1) into the cell nucleus in an amount sufficient to stimulate the cellular mitophagy in the subject.

In one embodiment a method of stimulating intracellular lipid biosynthesis in a cell nucleus of a subject in need thereof is provided, wherein the method comprises the step of: administering to the subject an effective amount of a sterol regulatory element-binding protein (SREBP) signaling pathway activator (or a pharmaceutically acceptable salt thereof) that induces nuclear translocation of SREBP N-terminal transactivation domain and neutral cholesterol ester hydrolase 1 (NCEH1) into the cell nucleus in an amount sufficient to stimulate the intracellular lipid synthesis in the subject.

In one embodiment a method of treating an intracellular mitophagy dysfunction-associated disease in a subject in need thereof is provided, wherein the method comprises the step of: administering to the subject an effective amount of a sterol regulatory element-binding protein (SREBP) signaling pathway activator, or a pharmaceutically acceptable salt thereof, that induces nuclear translocation of SREBP N-terminal transactivation domain and neutral cholesterol ester hydrolase 1 (NCEH1) into a cell nucleus.

In one embodiment a method to identify a biomarker for cellular mitophagy in a target tissue of a mammal is provided, wherein the method comprises the steps of:
(a) administering a sterol regulatory element-binding protein (SREBP) signaling pathway activator that induces SREBP C-terminal regulatory domain translocation into the cell nucleus or a pharmaceutically acceptable salt thereof, to the mammal;
(b) collecting endothelial cells from a blood sample of the mammal;
(c) analyzing the collected mammalian endothelial cells for cellular mitophagy;
(d) collecting cells from the target tissue of the mammal;
(e) analyzing the collected mammalian target tissue cells for cellular mitophagy; and
(f) determining a correlation between cellular mitophagy in the mammalian endothelial cells and cellular mitophagy in the mammalian target tissue cells.

In one embodiment a method of identifying a candidate sterol regulatory element-binding protein (SREBP) signaling pathway activator is provided, wherein the method comprising the steps of:
(a) contacting a cell with the candidate SREBP signaling pathway activator; and,
(b) analyzing the contacted cell for mitophagy and for presence of SREBP C-terminal regulatory domain or for presence of SREBP N-terminal transactivation domain and neutral cholesterol ester hydrolase 1 (NCEH1) translocation in the cell nucleus.

In one embodiment a method of stimulating intracellular mitophagy in a cell of a subject in need thereof is provided, wherein the method comprises the step of: administering to the subject an effective amount of propranolol or a pharmaceutically acceptable salt thereof sufficient to stimulate the cellular mitophagy in the subject.

In one embodiment a method of stimulating intracellular lipid biosynthesis in a cell nucleus of a subject in need thereof is provided, wherein the method comprising the step of: administering to the subject an effective amount of propranolol or a pharmaceutically acceptable salt thereof sufficient to stimulate the intracellular lipid synthesis in the subject.

Pharmaceutical compositions comprising SREBP signaling pathway activators, or pharmaceutically acceptable salts thereof, may be prepared with one or more pharmaceutically acceptable excipients which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 6th edition, American Pharmacists Association, 2009. Excipients can include ascorbic acid and other antioxidants, chelating agents such as EDTA, carbohydrates such as dextrin, hydroxyalkylcellulose, hydroxyalkylmethylcellulose, stearic acid and the like. In certain embodiments, the composition is provided as a solid dosage form, including a solid oral dosage form.

The compositions include those suitable for various administration routes, including oral administration. The compositions may be presented in unit dosage form and may be prepared by any of the methods well known in the art of pharmacy. Such methods include the step of bringing into association the active ingredient (e.g., a SREBP signaling pathway activator or a pharmaceutical salt thereof) with one or more pharmaceutically acceptable excipients. The compositions may be prepared by uniformly and intimately bringing into association the active ingredient with liquid excipients or finely divided solid excipients or both, and then, if necessary, shaping the product. Techniques and formulations generally are found in Remington: The Science and Practice of Pharmacy, 21^{st} Edition, Lippincott Wiliams and Wilkins, Philadelphia, Pa., 2006.

Compositions that are suitable for oral administration may be presented as discrete units (a unit dosage form) including but not limited to capsules, cachets or tablets each containing a predetermined amount of the active ingredient. In one embodiment, the pharmaceutical composition is a tablet.

Pharmaceutical compositions comprise one or more SREBP signaling pathway activators, or a pharmaceutically acceptable salt thereof, together with a pharmaceutically acceptable excipient and optionally other therapeutic agents. Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared.

Compositions intended for oral use may be prepared according to any method known to the art for the manufacture of pharmaceutical compositions and such compositions may contain one or more excipients including sweetening agents, flavoring agents, coloring agents and preserving agents, in order to provide a palatable preparation. Tablets containing the active ingredient in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 to 1000 mg of active material formulated with an appropriate and convenient amount of a pharmaceutically acceptable excipient. In certain embodiments, the pharmaceutically acceptable excipient varies from about 5 to about 95% of the total compositions (weight:weight).

In certain embodiments, a composition comprising a SREBP signaling pathway activator, or a pharmaceutically acceptable salt thereof, is provided wherein in one variation does not contain an agent that affects the rate at which the active ingredient is metabolized. Thus, it is understood that compositions comprising a SREBP signaling pathway activator in one aspect do not comprise an agent that would affect (e.g., slow, hinder or retard) the metabolism of a SREBP signaling pathway activator or any other active ingredient administered separately, sequentially or simultaneously with a SREBP signaling pathway activator. It is also understood that any of the methods, kits, articles of manufacture and the like detailed herein in one aspect do not comprise an agent that would affect (e.g., slow, hinder or retard) the metabolism of a SREBP signaling pathway activator or any other active ingredient administered separately, sequentially or simultaneously with a SREBP signaling pathway activator of the present disclosure.

SREBP signaling pathway activators can be administered with one or more additional therapeutic agent(s) to an individual (e.g. a human). Further, in certain embodiments, a SREBP signaling pathway activator of the present disclosure may be administered with one or more (e.g. one, two, three, four or more) additional therapeutic agent(s).

The SREBP signaling pathway activators of the present disclosure (also referred to herein as the active ingredients), can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient. An advantage of certain SREBP signaling pathway activators is that they are orally bioavailable and can be dosed orally.

A SREBP signaling pathway activator may be administered to an individual in accordance with an effective dosing regimen for a desired period of time or duration, such as at least about one month, at least about 2 months, at least about 3 months, at least about 6 months, or at least about 12 months or longer. In one variation, the SREBP signaling pathway activator is administered on a daily or intermittent schedule for the duration of the individual's life.

The dosage or dosing frequency of a SREBP signaling pathway activator may be adjusted over the course of the treatment, based on the judgment of the administering physician. The SREBP signaling pathway activator may be administered to an individual (e.g., a human) in an effective amount. In certain embodiments, the SREBP signaling pathway activator is administered once daily.

The SREBP signaling pathway activator can be administered by any useful route and means, such as by oral or parenteral (e.g., intravenous) administration. Therapeutically effective amounts of the SREBP signaling pathway activator may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A SREBP signaling pathway activator of the present disclosure may be combined with one or more additional therapeutic agents in any dosage amount of the SREBP signaling pathway activator (e.g., from 1 mg to 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the SREBP signaling pathway activator are about 100, 125, 150, 175, 200, 225, 250, 275, 300, 325, 350, 375, 400, 425, 450, 475, or about 500 mg per dose. Other therapeutically effective amounts of SREBP signaling pathway activators are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. Other therapeutically effective amounts of the SREBP signaling pathway activator are about 20, 25, 30, 35, 40, 45, 50, 55, 60, 65, 70, 75, 80, 85, 90, 95, or about 100 mg per dose.

The frequency of dosage of the SREBP signaling pathway activator of the present disclosure will be determined by the needs of the individual patient and can be, for example, once per day or twice, or more times, per day. Administration of the SREBP signaling pathway activator continues for as long as necessary to achieve the desired results. For example, a SREBP signaling pathway activator can be administered to a human being for a period of from 20 days to 180 days or, for example, for a period of from 20 days to 90 days or, for example, for a period of from 30 days to 60 days.

Administration can be intermittent, with a period of several or more days during which a patient receives a daily dose of the SREBP signaling pathway activator followed by a period of several or more days during which a patient does not receive a daily dose of the SREBP signaling pathway activator. For example, a patient can receive a dose of the SREBP signaling pathway activator every other day, or three times per week. Again, by way of example, a patient can receive a dose of the SREBP signaling pathway activator each day for a period of from 1 to 14 days, followed by a period of 7 to 21 days during which the patient does not receive a dose of the SREBP signaling pathway activator, followed by a subsequent period (e.g., from 1 to 14 days) during which the patient again receives a daily dose of the SREBP signaling pathway activator. Alternating periods of administration of the SREBP signaling pathway activator, followed by non-administration of the SREBP signaling pathway activator, can be repeated as clinically required to treat the patient.

In one embodiment, pharmaceutical compositions comprising a SREBP signaling pathway activator of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In one embodiment, kits comprising a compound of the present disclosure, or a pharmaceutically acceptable salt thereof, in combination with one or more (e.g., one, two, three, four, one or two, one to three, or one to four) additional therapeutic agents are provided.

In certain embodiments, a SREBP signaling pathway activator, or a pharmaceutically acceptable salt thereof, is combined with one, two, three, four or more additional therapeutic agents. In certain embodiments, a SREBP signaling pathway activator, or a pharmaceutically acceptable salt thereof, is combined with two additional therapeutic agents. In other embodiments, a SREBP signaling pathway activator, or a pharmaceutically acceptable salt thereof, is combined with three additional therapeutic agents. In further embodiments, a SREBP signaling pathway activator, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In certain embodiments, when a SREBP signaling pathway activator is combined with one or more additional therapeutic agents as described herein, the components of the composition are administered as a simultaneous or sequential regimen. When administered sequentially, the combination may be administered in two or more administrations.

In certain embodiments, a SREBP signaling pathway activator is combined with one or more additional therapeutic agents in a unitary dosage form for simultaneous administration to a patient, for example as a solid dosage form for oral administration.

In certain embodiments, a SREBP signaling pathway activator of the present disclosure is co-administered with one or more additional therapeutic agents.

The present disclosure provides a kit comprising a SREBP signaling pathway activator of the present disclosure or a pharmaceutically acceptable salt thereof. The kit may further comprise instructions for use. The instructions for use are generally written instructions, although electronic storage media (e.g., magnetic diskette or optical disk) containing instructions are also acceptable.

The present disclosure also provides a pharmaceutical kit comprising one or more containers comprising a SREBP signaling pathway activator or a pharmaceutically acceptable salt thereof. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the SREBP signaling pathway activators and instructions for use and be packaged in quantities sufficient for storage and use in pharmacies (e.g., hospital pharmacies and compounding pharmacies).

Also provided are articles of manufacture comprising a unit dosage of a SREBP signaling pathway activator or a pharmaceutically acceptable salt thereof, in suitable packaging for use in the methods described herein. Suitable packaging is known in the art and includes, for example, vials, vessels, ampules, bottles, jars, flexible packaging and the like. An article of manufacture may further be sterilized and/or sealed.

Inhibitors, such as NCEH1 inhibitors include, but are not limited to, agents such as microRNA, shRNA, siRNA, antisense, or ribozyme molecules specifically targeted to a nucleic acid molecule encoding NCEH1. Such agents can be designed based upon routine guidelines well-known in the art.

Inhibitors, such as inhibitors of NCEH1 gene expression for use in the present invention may be based on antisense oligonucleotide constructs. Anti-sense oligonucleotides, including anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell to recapitulate the phenotype of mitophagy induction and/or SREBP1-mediated lipid synthesis.

For example, antisense oligonucleotides of at least about 15 bases and complementary to unique regions of the mRNA transcript sequence encoding the target protein can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the targeted mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes may be delivered in vivo alone or in association with a vector. In its broadest sense, a “vector” is any vehicle capable of facilitating the transfer of the antisense oligonucleotide siRNA or ribozyme nucleic acid to the cells.

Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses;

herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular

Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBluescript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Aspects of the present disclosure can be described as embodiments in any of the following enumerated clauses. It will be understood that any of the described embodiments can be used in connection with any other described embodiments to the extent that the embodiments do not contradict one another.
Clause 1. A method to stimulate intracellular mitophagy in a cell nucleus of a subject in need thereof, the method comprising the step of: administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the cellular mitophagy in the subject.
Clause 2. The method according to the preceding clause 1, where the administered SREBP signaling pathway activator induces nuclear translocation of SREBP N-terminal transactivation domain linked to the SREBP C-terminal regulatory domain.
Clause 3. The method according to any one of clauses 1 or 2, where the administered SREBP signaling pathway activator induces nuclear translocation of intact, full length SREBP.
Clause 4. The method according to any one of clauses 1-3, wherein the administered SREBP signaling pathway activator further induces nuclear translocation of neutral cholesterol ester hydrolase 1 (NCEH1) into the cell nucleus and where the neutral cholesterol ester hydrolase 1 (NCEH1) is bound to the SREBP at the N-terminal transactivation domain.
Clause 5. The method according to any one of clauses 1-4, where the SREBP is an isoform selected from the group consisting of SREBP1a, SREBP1c and SREBP2.
Clause 6. The method according to any one of preceding clauses 1-5, where the SREBP signaling pathway activator that induces nuclear translocation of SREBP N-terminal transactivation domain and SREBP C-terminal regulatory domain into the cell nucleus is a neutral cholesterol ester hydrolase 1 (NCEH1) inhibitor.
Clause 7. The method according to any one of preceding clauses 1-6, where the mitophagy is whole mitochondrial autophagy.
Clause 8. The method according to any one of preceding clauses 1-7, where the mitophagy is ubiquitin-dependent mitophagy.
Clause 9. The method according to any one of preceding clauses 1-7, where the mitophagy is ubiquitin-independent mitophagy.
Clause 10. The method according to any one of preceding clauses 1-7, where the mitophagy is a canonical pathway-mediated mitophagy.
Clause 11. The method according to clause 10, where the intracellular mitophagy is a canonical pathway-mediated mitophagy selected from the group consisting of PTEN-induced putative kinase 1 (PINK1)/Parkin-mediated mitophagy, BCL2/adenovirus E1B 19 kDa protein-interacting protein 3/BNIP3-like protein (BNIP3/NIX)-mediated mitophagy, and FUN14 Domain Containing 1 (FUNDC1)-mediated mitophagy.
Clause 12. The method according to any one of preceding clauses 1-7, where the intracellular mitophagy is a non-canonical pathway-mediated mitophagy.
Clause 13. The method according to clause 12, where the intracellular mitophagy is a non-canonical pathway-mediated mitophagy selected from the group consisting of lipid-mediated mitophagy, Autophagy And Beclin 1 Regulator 1 (AMBRA1)-mediated mitophagy, BCL2 Like 13 (BCL2L13)-mediated mitophagy, FK506-binding protein 8 (FKBP8)-mediated mitophagy, and Rab-mediated mitophagy.
Clause 14. The method according to any one of preceding clauses 1-13, where the administered SREBP signaling pathway activator further stimulates intracellular lipid biosynthesis.
Clause 15. The method according to clause 14, where the stimulated intracellular lipid biosynthesis is intracellular cholesterol biosynthesis.
Clause 16. The method according to clause 14, where the stimulated intracellular lipid biosynthesis is intracellular unsaturated fatty acid biosynthesis.
Clause 17. The method according to any one of preceding clauses 14-16, where the stimulated intracellular lipid biosynthesis is accompanied by a change in a biomarker that corresponds to an increase in one or more intracellular lipids.
Clause 18. The method according to any one of the preceding clauses 1-17, where the subject is selected from the group consisting of mouse, rat, guinea pig, cat, dog, monkey, horse and human.
Clause 19. The method according to any one of preceding clauses 1-18, where the subject has a disease where decreased or impaired mitochondrial function is responsible for a symptom of the disease.
Clause 20. The method according to any one of preceding clauses 1-19, where the subject has a disease where decreased or impaired mitochondria function is wholly responsible for a symptom of the disease.
Clause 21. The method according to any one of preceding clauses 1-20, where the stimulated intracellular mitophagy is accompanied by a change in a biomarker that corresponds to an increase in mitochondrial clearance, production of new fully functional mitochondria in conjunction with clearing damaged mitochondria, and/or an increase in net intracellular mitochondrial function.
Clause 22. The method according to any one of preceding clauses 1-21, where the stimulated intracellular mitophagy clears damaged mitochondria and reduces reactive oxygen species (ROS).
Clause 23. The method according to any one of preceding clauses, where the stimulated intracellular mitophagy is accompanied by a change in a biomarker that 1-22 corresponds to an increase in mitochondrial clearance that is selected from the group consisting of decreased mitochondrial protein expression, decreased mitochondria DNA, or transmission electron microscopy of a cell.
Clause 24. The method according to any of the preceding clauses, where the stimulated intracellular mitophagy is accompanied by a change in a biomarker that is mitochondrial protein expression and the protein is TOM20, LC3 and/or P62.
Clause 25. The method according to any of the preceding clauses, where the subject has a disease and the stimulated intracellular mitophagy is accompanied by a change in a biomarker that corresponds to a decrease in a severity of a disease.
Clause 26. The method according to any of the preceding clauses, where the intracellular mitophagy is stimulated in a non-regenerative cell or tissue.
Clause 27. The method according to any one of preceding clauses 1-25, where the intracellular mitophagy is stimulated in a cell selected from the group consisting of endothelial cells, epithelial cells, smooth muscle cells, HEK293 cells, cardiomyocytes, liver cells, neurons, skeletal muscle, immune cells, and fibroblasts.
Clause 28. The method according to any of the preceding clauses, where the subject has a disease selected from the group consisting of ageing, genetic disease (e.g., X-linked IFAP), mitochondrial disease, lung/respiratory disease, cardiovascular disease, liver disease, renal disease, sepsis neurodegenerative/neuropsychiatric disease, and cancer.
Clause 29. The method according to any one of preceding clauses 1-27, where the subject has a disease selected from the group consisting of inflammatory diseases, eye diseases, auto-immune diseases, gastrointestinal diseases (peritonitis, inflammatory bowel disorders), and skeletal muscle conditions.
Clause 30. The method according to any one of preceding clauses 1-27, where the subject has a mitochondrial disease.
Clause 31. The method according to any one of preceding clauses 1-27, where the subject has a mitochondrial disease selected from the group consisting of Alpers Disease (Progressive Infantile Poliodystrophy), Barth Syndrome/LIC (Lethal Infantile Cardiomyopathy), Carnitine-Acyl-Carnitine Deficiency, Carnitine Deficiency, Co-Enzyme Q10 Deficiency, Mitochondrial Respiratory Chain Disorders, Complex I Deficiency, Complex II Deficiency, Complex III Deficiency, Complex IV/COX Deficiency, Complex V Deficiency, CPEO (Chronic Progressive External Ophthalmoplegia Syndrome), CPT I Deficiency, CPT II Deficiency, KSS (Kearns-Sayre Syndrome), Lactic Acidosis, LCAD (Long-Chain Acyl-CoA Dehydrogenase Deficiency) LCHAD, Leigh Disease or Syndrome (Subacute Necrotizing Encephalomyelopathy), Leber Hereditary Optic Neuropathy (LHON), Luft Disease, MAD/Glutaric Aciduria Type II, Maternally Inherited Deafness and Diabetes (MIDD), Multiple Acyl-CoA Dehydrogenase Deficiency (MADD), Medium Chain Acyl-CoA Dehydrogenase Deficiency (MCAD), Myoclonic Epilepsy and Ragged Red Fiber Disease (MERRF), Mitochondrial Cytopathy, Mitochondrial DNA Depletion, Mitochondrial Encephalomyopathy, Mitochondrial Myopathy, Mitochondrial Neurogastrointestinal Encephalomyopathy (MINGIE), Neuropathy Ataxia and Retinitis Pigmentosa (NARP), Pearson Syndrome, Pyruvate Carboxylase Deficiency, Pyruvate Dehydrogenase Deficiency, Short-Chain Acyl-CoA Dehydrogenase Deficiency (SCAD), Short-Chain 3-Hydroxyacyl-CoA Dehydrogenase Deficiency (SCHAD), and Very Long-Chain Acyl-CoA Dehydrogenase Deficiency (VLCAD).
Clause 32. The method according to any one of preceding clauses 1-27, where the subject has a mitochondrial disease and at least one symptom selected from the group consisting of muscle weakness, exercise intolerance, chronic fatigue, gastrointestinal dysmotility, impaired balance, vision loss, eye muscle and eyelid weakness, hearing loss, failure to thrive, over or underweight, developmental delay, neurodevelopmental regression, cognitive decline and memory impairment.
Clause 33. The method according to any one of preceding clauses 1-32, where the subject has a lung/respiratory disease.
Clause 34. The method according to clause 33, where the subject has a lung/respiratory disease selected from the group consisting of bronchopulmonary dysplasia, chronic obstructive pulmonary disease, emphysema, lung cancer, asthma, cystic fibrosis, pulmonary arterial hypertension, inflammatory lung disease, pleural cavity disease, pulmonary vascular disease, pneumonia, pulmonary embolism, idiopathic pulmonary fibrosis, interstitial lung disease, acute respiratory distress syndrome, sarcoidosis, mixed connective tissue disease, polymyositis, dermatomyositis, and systemic lupus erythematosus.
Clause 35. The method according to any of the preceding clauses, where the subject has a lung/respiratory disease that is pulmonary arterial hypertension.
Clause 36. The method according to any one of preceding clauses 1-27, where the subject has a cardiovascular disease.
Clause 37. The method according to clause 36, where the subject has a cardiovascular disease that is selected from the group consisting of acute cardiac ischemic events, acute myocardial infarction, angina, arrhythmia (atrial or ventricular), atherosclerosis, atrial fibrillation, atherosclerosis, arterial fibrillation, ischemic heart disease, cardiac insufficiency, cardiomyopathy (reduced and preserved ejection fraction), cardiovascular disease, chronic heart failure, chronic stable angina, congenital heart disease, congestive heart failure, coronary artery bypass grafting, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mitophagy in cardiac surgery, mixed dyslipidemia, moderate to mild heart failure, myocardial infarction, myocarditis, obesity management, paroxysmal atrial/arterial fibrillation/flutter, paroxysmal supraventricular tachycardias (PSVT), particularly severe or rapid onset edema, peripheral vascular disease, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.
Clause 38. The method according to any one of preceding clauses 36-37, where the subject has a cardiovascular disease that is hypertension.
Clause 39. The method according to any one of preceding clauses 1-27, where the subject has a liver disease.
Clause 40. The method according to clause 39, where the subject has a liver disease that is selected from the group consisting of alcoholic liver disease, fatty liver disease, non-alcoholic fatty liver disease (NAFLD), non-alcoholic steatohepatitis (NASH), alcoholic hepatitis, liver cancer, hepatic fibrosis, liver ischemia/reperfusion injury, primary sclerosing cholangitis, primary biliary cholangitis, viral hepatitis (Chronic Hepatitis C, HBV), autoimmune hepatitis, iatrogenic and/or drug induced liver injury, steatosis, and hepatic veno-occlusive disease.
Clause 41. The method according to any one of preceding clauses 1-27, where the subject has a liver disease that is non-alcoholic fatty liver disease.
Clause 42. The method according to any one of preceding clauses 1-27, where the subject has a renal disease.
Clause 43. The method according to clause 42, where the renal disease is selected from the group consisting of kidney failure, acute kidney injury, chronic kidney disease, diabetic nephropathy, end stage renal disease, interstitial nephritis, kidney fibrosis, tubulointerstitial fibrosis, severe interstitial fibrosis, renal dysfunction, renal interstitial fibrosis, renal failure, and polycystic kidney disease.
Clause 44. The method according to any one of preceding clauses 42-43, where the subject has a renal disease that is acute kidney injury.
Clause 45. The method according to any one of preceding clauses 1-27, where the subject has a neurodegenerative/neuropsychiatric disease.
Clause 46. The method according to clause 45, where the neurodegenerative/neuropsychiatric disease is selected from the group consisting of amyotrophic lateral sclerosis, bipolar disorder, autism, catalepsy, cerebrovascular disease, depression, dementia, epilepsy, encephalitis, meningitis, migraine, Huntington's, Alzheimer's, Parkinson's, post-stroke cognitive impairment, schizophrenia, traumatic brain injury, and multiple sclerosis.
Clause 47. The method according to clause 46, where the subject has a neurodegenerative/neuropsychiatric disease that is Parkinson's disease.
Clause 48. The method according to any one of preceding clauses 1-27, where the subject has a cancer.
Clause 49. The method according to clause 48, where the subject has a cancer that is selected from the group consisting of acute lymphoblastic leukemia, acute myeloid leukemia, adenoid cystic carcinoma, ampullary carcinoma, bladder cancer, breast cancer, cervical cancer, cholangiocarcinoma, clear cell renal cell carcinoma, colorectal cancer, cutaneous melanoma, ependydoma, esophageal cancer, esophageal squamous cell carcinoma, gastric cancer, gastric carcinoma, gastric adenocarcinoma, glioblastoma, glioma, head and neck squamous cell carcinoma, hepatocellular carcinoma, laryngeal cancer, laryngeal squamous cell carcinoma, leukemia, locally advanced rectal cancer, lung cancer, lymphoma, melanoma, multiple myeloma, non-small cell lung cancer, oral melanoma, oral squamous cell carcinoma, oropharyngeal squamous cell carcinoma, osteosarcoma, ovarian cancer, pancreatic cancer, pancreatic ductal adenocarcinoma, prostate cancer, renal carcinoma, squamous cell carcinoma, tongue squamous cell carcinoma, triple negative breast cancer, and uterine-cervical squamous cell carcinoma.
Clause 50. The method according to clause 48, where the subject has a cancer that is lung cancer.
Clause 51. The method according to any of the preceding clauses, where the subject has a disease selected from the group consisting of hemorrhoids (internal, external), varices (esophageal, bowel, gastric), arterio-venous malformations (AVMs), cavernous malformations, telangiectasias, plexiform lesions, endotheliomas, hemangiomas (benign, infantile, hepatic, cerebral, pulmonary, congenital, other organs), pyogenic granuloma, hemangioendothelioma (Kaposiform, epithelioid), angiosarcoma, capillary malformations, venous malformations, arterial malformations, arterio-venous fistula and veno-lymphatic malformations.
Clause 52. The method according to any of the preceding clauses, where the SREBP signaling pathway activator is a small molecule SREBP signaling pathway activator.
Clause 53. The method according to any of the preceding clauses, where the SREBP signaling pathway activator is a selective SREBP signaling pathway activator.
Clause 54. The method according to any of the preceding clauses, where the SREBP signaling pathway activator increases expression of a gene encoding SREBP.
Clause 55. The method according to any of the preceding clauses, where the SREBP signaling pathway activator is an NCEH1 inhibitor that blocks enzymatic activity of NCEH1.
Clause 56. The method according to any of the preceding clauses, where NCEH1 inhibition activates both canonical and non-canonical SREBP signaling pathways
Clause 57. The method according to any of the preceding clauses, where NCEH1 inhibition induces nuclear translocation of NCEH1 bound to the SREBP N-terminal transactivation domain.
Clause 58. The method according to any of the preceding clauses, where the SREBP pathway activation is contingent upon nuclear translocation of NCEH1.
Clause 59. The method according to any of the preceding clauses, where the SREBP signaling pathway activator is an NCEH1 inhibitor that reduces expression of a gene encoding NCEH1.
Clause 60. The method according to any of the preceding clauses, where the SREBP signaling pathway activator is an NCEH1 inhibitor that is a siRNA, an antisense oligonucleotide or a ribozyme.
Clause 61. The method according to any of the preceding clauses, where the SREBP signaling pathway activator is an NCEH1 inhibitor that is a small molecule NCEH1 inhibitor.
Clause 62. The method according to any of the preceding clauses, where the SREBP signaling pathway activator is a nonselective NCEH1 inhibitor that is selected from the group consisting of propranolol, R-propranolol, papuamide, U18666A and JW480 or a pharmaceutically acceptable salt of propranolol, R-propranolol, papuamide, U18666A and JW480.
Clause 63. The method according to any of the preceding clauses, where the SREBP signaling pathway activator is an NCEH1 inhibitor that is a selective NCEH1 inhibitor.
Clause 64. The method according to any of the preceding clauses, where the SREBP signaling pathway activator is a selective NCEH1 inhibitor that is JW480.
Clause 65. The method according to any of the preceding clauses, where the SREBP signaling pathway activator is co-administered with one or more other therapeutic agents.
Clause 66. A method to stimulate intracellular lipid biosynthesis in a cell nucleus of a subject in need thereof, the method comprising the step of: administering to the subject an effective amount of a sterol regulatory element-binding protein (SREBP) signaling pathway activator that induces nuclear translocation of SREBP C-terminal regulatory domain into the cell nucleus or a pharmaceutically acceptable salt thereof sufficient to stimulate the intracellular lipid synthesis in the subject.
Clause 67. The method according to clause 66, where the administered SREBP signaling pathway activator induces nuclear translocation of SREBP N-terminal transactivation domain linked to the SREBP C-terminal regulatory domain.
Clause 68. The method according to any one of preceding clauses 66-67, where the administered SREBP signaling pathway activator induces nuclear translocation of intact, full length SREBP.
Clause 69. The method according to any one of preceding clauses 66-68, where the administered SREBP signaling pathway activator further induces nuclear translocation of neutral cholesterol ester hydrolase 1 (NCEH1) into the cell nucleus and where the neutral cholesterol ester hydrolase 1 (NCEH1) is bound to the SREBP at the N-terminal transactivation domain.
Clause 70. The method according to any one of preceding clauses 66-69, where the SREBP is an isoform selected from the group consisting of SREBP1a, SREBP1c and SREBP2.
Clause 71. The method according to any one of preceding clauses 66-70, where the SREBP signaling pathway activator that induces SREBP C-terminal regulatory domain translocation into the cell nucleus is a neutral cholesterol ester hydrolase 1 (NCEH1) inhibitor.
Clause 72. The method according to any one of preceding clauses 66-71, where the stimulated intracellular lipid biosynthesis reduces free cholesterol.
Clause 73. The method according to any one of preceding clauses 66-72, where the administered SREBP signaling pathway activator further stimulates intracellular mitophagy.
Clause 74. The method according to any one of preceding clauses 66-73, where administered SREBP signaling pathway activator further stimulates intracellular mitophagy and where the stimulated intracellular mitophagy is accompanied by a change in a biomarker that corresponds to an increase in mitochondrial clearance that is selected from the group consisting of decreased mitochondrial protein expression, decreased mitochondria DNA, or transmission electron microscopy of a cell.
Clause 75. The method according to any one of preceding clauses 66-74, where the stimulated intracellular mitophagy is accompanied by a change in a biomarker that is mitochondrial protein expression and the protein is TOM20, LC3 and/or P62.
Clause 76. The method according to any one of preceding clauses 66-75, where the intracellular mitophagy is whole mitochondrial autophagy.
Clause 77. The method according to any of the preceding clauses 66-76, where the intracellular mitophagy is ubiquitin-dependent mitophagy.
Clause 78. The method according to any of the preceding clauses 66-76, where the intracellular mitophagy is ubiquitin-independent mitophagy.
Clause 79. The method according to any of the preceding clauses 66-76, where the intracellular mitophagy is a canonical pathway-mediated mitophagy.
Clause 80. The method according to any of the preceding clauses 66-76, where the intracellular mitophagy is a canonical pathway-mediated mitophagy selected from the group consisting of PTEN-induced putative kinase 1 (PINK1)/Parkin-mediated mitophagy, BCL2/adenovirus E1B 19 kDa protein-interacting protein 3/BNIP3-like protein (BNIP3/NIX)-mediated mitophagy, and FUN14 Domain Containing 1 (FUNDC1)-mediated mitophagy.
Clause 81. The method according to any of the preceding clauses 66-76, where the intracellular mitophagy is a non-canonical pathway-mediated mitophagy.
Clause 82. The method according to any of the preceding clauses 66-76, where the intracellular mitophagy is a non-canonical pathway-mediated mitophagy selected from the group consisting of lipid-mediated mitophagy, Autophagy And Beclin 1 Regulator 1 (AMBRA1)-mediated mitophagy, BCL2 Like 13 (BCL2L13)-mediated mitophagy, FK506-binding protein 8 (FKBP8)-mediated mitophagy, and Rab-mediated mitophagy.
Clause 83. The method according to any of the preceding clauses 66-82, where the stimulated intracellular lipid biosynthesis is accompanied by a change in a biomarker that corresponds to an increase in one or more intracellular lipids.
Clause 84. The method according to any of the preceding clauses 66-82, where the stimulated intracellular lipid biosynthesis is accompanied by a change in a biomarker that corresponds to an increase in mitochondrial clearance, production of new fully functional mitochondria in conjunction with clearing damaged mitochondria, and/or an increase in net cellular mitochondrial function.
Clause 85. The method according to any of the preceding clauses 66-82, where the subject has a disease and the stimulated intracellular lipid biosynthesis is accompanied by a change in a biomarker that corresponds to a decrease in a severity of a disease.
Clause 86. The method according to clause 85, where intracellular lipid biosynthesis is stimulated in a non-regenerative cell or tissue.
Clause 87. The method according to any of the preceding clauses 85-86, where the intracellular lipid biosynthesis is stimulated in a cell selected from the group consisting of endothelial cells, smooth muscle cells, HEK293 cells, cardiomyocytes, liver cells, neurons, skeletal muscle and fibroblasts.
Clause 88. The method according to any of the preceding clauses 66-87, where the subject has a disease selected from the group consisting of mitochondrial disease, lung/respiratory disease, cardiovascular disease, liver disease, renal disease, neurodegenerative/neuropsychiatric disease and cancer.
Clause 89. The method according to any of the preceding clauses 66-87, where the subject has a mitochondrial disease.
Clause 90. The method according to any of the preceding clauses 66-87, where the subject has a mitochondrial disease selected from the group consisting of Alpers Disease (Progressive Infantile Poliodystrophy), Barth Syndrome/LIC (Lethal Infantile Cardiomyopathy), Carnitine-Acyl-Carnitine Deficiency, Carnitine Deficiency, Co-Enzyme Q10 Deficiency, Mitochondrial Respiratory Chain Disorders, Complex I Deficiency, Complex II Deficiency, Complex III Deficiency, Complex IV/COX Deficiency, Complex V Deficiency, CPEO (Chronic Progressive External Ophthalmoplegia Syndrome), CPT I Deficiency, CPT II Deficiency, KSS (Kearns-Sayre Syndrome), Lactic Acidosis, LCAD (Long-Chain Acyl-CoA Dehydrogenase Deficiency) LCHAD, Leigh Disease or Syndrome (Subacute Necrotizing Encephalomyelopathy), LHON (Leber Hereditary Optic Neuropathy), Luft Disease, MAD/Glutaric Aciduria Type II, Maternally Inherited Deafness and Diabetes (MIDD), Multiple Acyl-CoA Dehydrogenase Deficiency (MADD), Medium Chain Acyl-CoA Dehydrogenase Deficiency (MCAD), Myoclonic Epilepsy and Ragged Red Fiber Disease (MERRF), Mitochondrial Cytopathy, Mitochondrial DNA Depletion, Mitochondrial Encephalomyopathy, Mitochondrial Myopathy, Mitochondrial Neurogastrointestinal Encephalomyopathy (MINGIE), Neuropathy Ataxia and Retinitis Pigmentosa (NARP), Pearson Syndrome, Pyruvate Carboxylase Deficiency, Pyruvate Dehydrogenase Deficiency, Short-Chain Acyl-CoA Dehydrogenase Deficiency (SCAD), Short-Chain 3-Hydroxyacyl-CoA Dehydrogenase Deficiency (SCHAD), and Very Long-Chain Acyl-CoA Dehydrogenase Deficiency (VLCAD).
Clause 91. The method according to any of the preceding clauses 66-87, where the subject has a mitochondrial disease and at least one symptom selected from the group consisting of muscle weakness, exercise intolerance, chronic fatigue, gastrointestinal dysmotility, impaired balance, vision loss, eye muscle and eyelid weakness, hearing loss, failure to thrive, over or underweight, developmental delay, neurodevelopmental regression, cognitive decline and memory impairment.
Clause 92. The method according to any of the preceding clauses 66-87, where the subject has a lung/respiratory disease.
Clause 93. The method according to any of the preceding clauses 66-87, where the subject has a lung/respiratory disease selected from the group consisting of bronchopulmonary dysplasia, chronic obstructive pulmonary disease, lung cancer, asthma, cystic fibrosis, pulmonary arterial hypertension, inflammatory lung disease, pleural cavity disease, pulmonary vascular disease, pneumonia, pulmonary embolism, idiopathic pulmonary fibrosis, sarcoidosis, mixed connective tissue disease, polymyositis, dermatomyositis, and systemic lupus erythematosus.
Clause 94. The method according to any of the preceding clauses 66-87, where the subject has a lung/respiratory disease that is pulmonary arterial hypertension.
Clause 95. The method according to any of the preceding clauses 66-87, where the subject has a cardiovascular disease.
Clause 96. The method according to any of the preceding clauses 66-87, where the subject has a cardiovascular disease that is selected from the group consisting of acute cardiac ischemic events, acute myocardial infarction, angina, arrhythmia, atherosclerosis, atrial fibrillation, atherosclerosis, arterial fibrillation, ischemic heart disease, cardiac insufficiency, cardiomyopathy, cardiovascular disease, chronic heart failure, chronic stable angina, congenital heart disease, congestive heart failure, coronary artery bypass grafting, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mitophagy in cardiac surgery, mixed dyslipidemia, moderate to mild heart failure, myocardial infarction, obesity management, paroxysmal atrial/arterial fibrillation/flutter, paroxysmal supraventricular tachycardias (PSVT), particularly severe or rapid onset edema, peripheral vascular disease, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.
Clause 97. The method according to any of the preceding clauses 66-87, where the subject has a cardiovascular disease that is hypertension.
Clause 98. The method according to any of the preceding clauses 66-87, where the subject has a liver disease.
Clause 99. The method according to any of the preceding clauses 66-87, where the subject has a liver disease that is selected from the group consisting of alcoholic liver disease, fatty liver disease, non-alcoholic fatty liver disease (NAFLD), non-alcoholic steatohepatitis (NASH), alcoholic hepatitis, liver cancer, hepatic fibrosis, liver ischemia/reperfusion injury, primary sclerosing cholangitis, primary biliary cholangitis, viral hepatitis (Chronic Hepatitis C, HBV), autoimmune hepatitis, iatrogenic and/or drug induced liver injury, steatosis, and hepatic veno-occlusive disease.
Clause 100. The method according to any of the preceding clauses 66-87, where the subject has a liver disease that is non-alcoholic fatty liver disease.
Clause 101. The method according to any of the preceding clauses 66-87, where the subject has a renal disease.
Clause 102. The method according to any of the preceding clauses 66-87, where the renal disease is selected from the group consisting of kidney failure, acute kidney injury, chronic kidney disease, diabetic nephropathy, end stage renal disease, interstitial nephritis, kidney fibrosis, tubulointerstitial fibrosis, severe interstitial fibrosis, renal dysfunction, renal interstitial fibrosis, and polycystic kidney disease.
Clause 103. The method according to any of the preceding clauses 66-87, where the subject has a renal disease that is acute kidney injury.
Clause 104. The method according to any of the preceding clauses 66-87, where the subject has a neurodegenerative/neuropsychiatric disease.
Clause 105. The method according to any of the preceding clauses 66-87, where the neurodegenerative/neuropsychiatric disease is selected from the group consisting of amyotrophic lateral sclerosis, bipolar disorder, autism, catalepsy, depression, dementia, epilepsy, encephalitis, meningitis, migraine, Huntington's, Alzheimer's, Parkinson's, post-stroke cognitive impairment, schizophrenia, and multiple sclerosis.
Clause 106. The method according to any of the preceding clauses 66-87, where the subject has a neurodegenerative/neuropsychiatric disease is Parkinson's disease.
Clause 107. The method according to any of the preceding clauses 66-87, where the subject has a cancer.
Clause 108. The method according to any of the preceding clauses 66-87, where the subject has a cancer that is selected from the group consisting of acute lymphoblastic leukemia, acute myeloid leukemia, adenoid cystic carcinoma, ampullary carcinoma, bladder cancer, breast cancer, cervical cancer, cholangiocarcinoma, clear cell renal cell carcinoma, colorectal cancer, cutaneous melanoma, ependydoma, esophageal cancer, esophageal squamous cell carcinoma, gastric cancer, gastric carcinoma, gastric adenocarcinoma, glioblastoma, glioma, head and neck squamous cell carcinoma, hepatocellular carcinoma, laryngeal cancer, laryngeal squamous cell carcinoma, leukemia, locally advanced rectal cancer, lung cancer, lymphoma, melanoma, multiple myeloma, non-small cell lung cancer, oral melanoma, oral squamous cell carcinoma, oropharyngeal squamous cell carcinoma, osteosarcoma, ovarian cancer, pancreatic cancer, pancreatic ductal adenocarcinoma, prostate cancer, renal carcinoma, squamous cell carcinoma, tongue squamous cell carcinoma, triple negative breast cancer, and uterine-cervical squamous cell carcinoma.
Clause 109. The method according to any of the preceding clauses 66-87, where the subject has a cancer that is lung cancer.
Clause 110. The method according to any of the preceding clauses, where the SREBP signaling pathway activator is a small molecule SREBP signaling pathway activator.
Clause 111. The method according to any of the preceding clauses, where the SREBP signaling pathway activator is a selective SREBP signaling pathway activator.
Clause 112. The method according to any of the preceding clauses, where the SREBP signaling pathway activator increases expression of a gene encoding SREBP.
Clause 113. The method according to any of the preceding clauses, where the SREBP signally pathway activation is contingent upon nuclear translocation of NCEH1.
Clause 114. The method according to any of the preceding clauses, where the SREBP signaling pathway activator is an NCEH1 inhibitor that blocks enzymatic activity of NCEH1.
Clause 115. The method according to any of the preceding clauses, where the SREBP signaling pathway activator is an NCEH1 inhibitor that reduces expression of a gene encoding NCEH1.
Clause 116. The method according to any of the preceding clauses, where the SREBP signaling pathway activator is an NCEH1 inhibitor that is a siRNA, an antisense oligonucleotide or a ribozyme.
Clause 117. The method according to any of the preceding clauses, where the SREBP signaling pathway activator is an NCEH1 inhibitor that is a small molecule NCEH1 inhibitor.
Clause 118. The method according to any of the preceding clauses, where the SREBP signaling pathway activator is a nonselective NCEH1 inhibitor that is propranolol or R-propranolol.
Clause 119. The method according to any of the preceding clauses, where the SREBP signaling pathway activator is an NCEH1 inhibitor that is a selective NCEH1 inhibitor.
Clause 120. The method according to any of the preceding clauses, where the SREBP signaling pathway activator is a selective NCEH1 inhibitor that is JW480.
Clause 121. The method according to any of the preceding clauses, where the SREBP signaling pathway activator is co-administered with one or more other therapeutic agents.
Clause 122. A method to treat an intracellular mitophagy dysfunction-associated disease in a subject in need thereof, the method comprising the step of: administering to the subject an effective amount of a sterol regulatory element-binding protein (SREBP) signaling pathway activator that induces nuclear translocation of SREBP C-terminal regulatory domain into a cell nucleus or a pharmaceutically acceptable salt thereof.
Clause 123. The method according to the preceding clause 122, where the administered SREBP signaling pathway activator induces nuclear translocation of SREBP N-terminal transactivation domain linked to the SREBP C-terminal regulatory domain.
Clause 124. The method according to any of the preceding clauses 122-123, where the administered SREBP signaling pathway activator induces nuclear translocation of intact, full length SREBP.
Clause 125. The method according to any of the preceding clauses 122-124, where the administered SREBP signaling pathway activator further induces nuclear translocation of neutral cholesterol ester hydrolase 1 (NCEH1) into the cell nucleus and where the neutral cholesterol ester hydrolase 1 (NCEH1) is bound to the SREBP at the N-terminal transactivation domain.
Clause 126. The method according to any of the preceding clauses 122-125, where the SREBP is an isoform selected from the group consisting of SREBP1a, SREBP1c and SREBP2.
Clause 127. The method according to any of the preceding clauses 122-126, where the SREBP signaling pathway activator that induces nuclear translocation of SREBP N-terminal transactivation domain and SREBP C-terminal regulatory domain in the cell nucleus is a neutral cholesterol ester hydrolase 1 (NCEH1) inhibitor.
Clause 128. The method according to any of the preceding clauses 122-127, where the intracellular mitophagy is whole mitochondrial autophagy.
Clause 129. The method according to any of the preceding clauses 122-127, where the mitophagy dysfunction is ubiquitin-dependent mitophagy.
Clause 130. The method according to any of the preceding clauses 122-127, where the mitophagy dysfunction is ubiquitin-independent mitophagy.
Clause 131. The method according to any of the preceding clauses 122-127, where the mitophagy dysfunction is canonical pathway-mediated mitophagy.
Clause 132. The method according to any of the preceding clauses 122-127, where the mitophagy dysfunction is a canonical pathway-mediated mitophagy selected from the group consisting of PTEN-induced putative kinase 1 (PINK1)/Parkin-mediated mitophagy, BCL2/adenovirus E1B 19 kDa protein-interacting protein 3/BNIP3-like protein (BNIP3/NIX)-mediated mitophagy, and FUN14 Domain Containing 1 (FUNDC1)-mediated mitophagy.
Clause 133. The method according to any of the preceding clauses 122-127, where the mitophagy dysfunction is a non-canonical pathway-mediated mitophagy.
Clause 134. The method according to any of the preceding clauses 122-127, where the mitophagy dysfunction is a non-canonical pathway-mediated mitophagy selected from the group consisting of lipid-mediated mitophagy, Autophagy And Beclin 1 Regulator 1 (AMBRA1)-mediated mitophagy, BCL2 Like 13 (BCL2L13)-mediated mitophagy, FK506-binding protein 8 (FKBP8)-mediated mitophagy, and Rab-mediated mitophagy.
Clause 135. The method according to any of the preceding clauses 122-134, where the subject is selected from the group consisting of mouse, rat, guinea pig, cat, dog, monkey, horse and human.
Clause 136. The method according to any of the preceding clauses 122-135, where the mitophagy dysfunction is wholly responsible for the disease.
Clause 137. The method according to any of the preceding clauses 122-136, where the administered SREBP signaling pathway activator stimulates intracellular mitophagy.
Clause 138. The method according to any of the preceding clauses, where the administered SREBP signaling pathway activator stimulates intracellular lipid biosynthesis.
Clause 139. The method according to any of the preceding clauses, where the administered SREBP signaling pathway activator stimulates intracellular lipid biosynthesis and the stimulated intracellular lipid biosynthesis reduces free cholesterol.
Clause 140. The method according to any of the preceding clauses, where the administered SREBP signaling pathway activator stimulates intracellular lipid biosynthesis and the stimulated intracellular lipid biosynthesis is intracellular unsaturated fatty acid biosynthesis.
Clause 141. The method according to any of the preceding clauses, where the administered SREBP signaling pathway activator stimulates intracellular lipid biosynthesis and where the stimulated intracellular lipid biosynthesis is accompanied by a change in a biomarker that corresponds to an increase in one or more intracellular lipids.
Clause 142. The method according to any of the preceding clauses, where the treated mitophagy dysfunction-associated disease is accompanied by a change in a biomarker that corresponds to an increase in one or more intracellular lipids.
Clause 143. The method according to any of the preceding clauses, where the treated mitophagy dysfunction-associated disease is accompanied by a change in a biomarker that corresponds to a change in one or more intracellular lipids selected from the group consisting of a decrease in free cholesterol and an increase in newly made non-oxidized/non-damaged fatty acids.
Clause 144. The method according to any of the preceding clauses, where the treated mitophagy dysfunction-associated disease is accompanied by a change in a biomarker that corresponds to a decrease in free cholesterol.
Clause 145. The method according to any of the preceding clauses, where the treated mitophagy dysfunction-associated disease is accompanied by a change in a biomarker that corresponds to an increase in mitochondrial clearance, production of new fully functional mitochondria in conjunction with clearing damaged mitochondria, and/or an increase in net intracellular mitochondrial function.
Clause 146. The method according to any of the preceding clauses, where the stimulated intracellular mitophagy is accompanied by a change in a biomarker that corresponds to an increase in mitochondrial clearance that is selected from the group consisting of increased mitochondrial protein expression, increased mitochondrial DNA, or transmission electron microscopy of a cell.
Clause 147. The method according to any of the preceding clauses, where the stimulated intracellular mitophagy is accompanied by a change in a biomarker that is mitochondrial protein expression and the protein is TOM20, LC3 and/or P62.
Clause 148. The method according to any of the preceding clauses, where the treated mitophagy-dysfunction associated disease is accompanied by a change in a biomarker that corresponds to a decrease in a severity of a disease.
Clause 149. The method according to any of the preceding clauses, where the intracellular mitophagy is stimulated in a non-regenerative cell or tissue.
Clause 150. The method according to any of the preceding clauses, where the intracellular mitophagy is stimulated in a cell selected from the group consisting of endothelial cells, smooth muscle cells, HEK293 cells, cardiomyocytes, liver cells, neurons, skeletal muscle and fibroblasts.
Clause 151. The method according to any of the preceding clauses, where the mitophagy dysfunction-associated disease is selected from the group consisting of mitochondrial disease, lung/respiratory disease, cardiovascular disease, liver disease, renal disease, neurodegenerative/neuropsychiatric disease and cancer.
Clause 152. The method according to any of the preceding clauses, where the mitophagy dysfunction-associated disease is a mitochondrial disease.
Clause 153. The method according to any of the preceding clauses, where the mitophagy dysfunction-associated disease is a mitochondrial disease selected from the group consisting of Alpers Disease (Progressive Infantile Poliodystrophy), Barth Syndrome/LIC (Lethal Infantile Cardiomyopathy), Carnitine-Acyl-Carnitine Deficiency, Carnitine Deficiency, Co-Enzyme Q10 Deficiency, Mitochondrial Respiratory Chain Disorders, Complex I Deficiency, Complex II Deficiency, Complex III Deficiency, Complex IV/COX Deficiency, Complex V Deficiency, CPEO (Chronic Progressive External Ophthalmoplegia Syndrome), CPT I Deficiency, CPT II Deficiency, KSS (Kearns-Sayre Syndrome), Lactic Acidosis, LCAD (Long-Chain Acyl-CoA Dehydrogenase Deficiency) LCHAD, Leigh Disease or Syndrome (Subacute Necrotizing Encephalomyelopathy), LHON (Leber Hereditary Optic Neuropathy), Luft Disease, MAD/Glutaric Aciduria Type II, Maternally Inherited Deafness and Diabetes (MIDD), Multiple Acyl-CoA Dehydrogenase Deficiency (MADD), Medium Chain Acyl-CoA Dehydrogenase Deficiency (MCAD), Myoclonic Epilepsy and Ragged Red Fiber Disease (MERRF), Mitochondrial Cytopathy, Mitochondrial DNA Depletion, Mitochondrial Encephalomyopathy, Mitochondrial Myopathy, Mitochondrial Neurogastrointestinal Encephalomyopathy (MINGIE), Neuropathy Ataxia and Retinitis Pigmentosa (NARP), Pearson Syndrome, Pyruvate Carboxylase Deficiency, Pyruvate Dehydrogenase Deficiency, Short-Chain Acyl-CoA Dehydrogenase Deficiency (SCAD), Short-Chain 3-Hydroxyacyl-CoA Dehydrogenase Deficiency (SCHAD), and Very Long-Chain Acyl-CoA Dehydrogenase Deficiency (VLCAD).
Clause 154. The method according to any of the preceding clauses, where the mitophagy dysfunction-associated disease is accompanied by at least one symptom selected from the group consisting of muscle weakness, exercise intolerance, chronic fatigue, gastrointestinal dysmotility, impaired balance, vision loss, eye muscle and eyelid weakness, hearing loss, failure to thrive, over or underweight, developmental delay, neurodevelopmental regression, cognitive decline and memory impairment.
Clause 155. The method according to any of the preceding clauses, where the mitophagy dysfunction-associated disease is a lung/respiratory disease.
Clause 156. The method according to any of the preceding clauses, where the mitophagy dysfunction-associated disease is a lung/respiratory disease selected from the group consisting of bronchopulmonary dysplasia, chronic obstructive pulmonary disease, lung cancer, asthma, cystic fibrosis, pulmonary arterial hypertension, inflammatory lung disease, pleural cavity disease, pulmonary vascular disease, pneumonia, pulmonary embolism, idiopathic pulmonary fibrosis, sarcoidosis, mixed connective tissue disease, polymyositis, dermatomyositis, and systemic lupus erythematosus.
Clause 157. The method according to any of the preceding clauses, where the mitophagy dysfunction-associated disease is a lung/respiratory disease that is pulmonary arterial hypertension.
Clause 158. The method according to any of the preceding clauses, where the mitophagy dysfunction-associated disease is a cardiovascular disease.
Clause 159. The method according to any of the preceding clauses, where the mitophagy dysfunction-associated disease is a cardiovascular disease selected from the group consisting of acute cardiac ischemic events, acute myocardial infarction, angina, arrhythmia, atherosclerosis, atrial fibrillation, atherosclerosis, arterial fibrillation, ischemic heart disease, cardiac insufficiency, cardiomyopathy, cardiovascular disease, chronic heart failure, chronic stable angina, congenital heart disease, congestive heart failure, coronary artery bypass grafting, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mitophagy in cardiac surgery, mixed dyslipidemia, moderate to mild heart failure, myocardial infarction, obesity management, paroxysmal atrial/arterial fibrillation/flutter, paroxysmal supraventricular tachycardias (PSVT), particularly severe or rapid onset edema, peripheral vascular disease, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.
Clause 160. The method according to any of the preceding clauses, where the mitophagy dysfunction-associated disease is a cardiovascular disease that is hypertension.
Clause 161. The method according to any of the preceding clauses, where the mitophagy dysfunction-associated disease is a liver disease.
Clause 162. The method according to any of the preceding clauses, where the mitophagy dysfunction-associated disease is a liver disease selected from the group consisting of alcoholic liver disease, fatty liver disease, non-alcoholic fatty liver disease (NAFLD), non-alcoholic steatohepatitis (NASH), alcoholic hepatitis, liver cancer, hepatic fibrosis, liver ischemia/reperfusion injury, primary sclerosing cholangitis, primary biliary cholangitis, viral hepatitis (Chronic Hepatitis C, HBV), autoimmune hepatitis, iatrogenic and/or drug induced liver injury, steatosis, and hepatic veno-occlusive disease.
Clause 163. The method according to any of the preceding clauses, where the mitophagy dysfunction-associated disease is a liver disease that is non-alcoholic fatty liver disease.
Clause 164. The method according to any of the preceding clauses, where the mitophagy dysfunction-associated disease is a renal disease.
Clause 165. The method according to any of the preceding clauses, where the mitophagy dysfunction-associated disease is a renal disease selected from the group consisting of kidney failure, acute kidney injury, chronic kidney disease, diabetic nephropathy, end stage renal disease, interstitial nephritis, kidney fibrosis, tubulointerstitial fibrosis, severe interstitial fibrosis, renal dysfunction, renal interstitial fibrosis, and polycystic kidney disease.
Clause 166. The method according to any of the preceding clauses, where the mitophagy dysfunction-associated disease is a renal disease that is acute kidney injury.
Clause 167. The method according to any of the preceding clauses, where the mitophagy dysfunction-associated disease is a neurodegenerative/neuropsychiatric disease.
Clause 168. The method according to any of the preceding clauses, where the neurodegenerative/neuropsychiatric disease is selected from the group consisting of amyotrophic lateral sclerosis, bipolar disorder, autism, catalepsy, depression, dementia, epilepsy, encephalitis, meningitis, migraine, Huntington's, Alzheimer's, Parkinson's, post-stroke cognitive impairment, schizophrenia, and multiple sclerosis.
Clause 169. The method according to any of the preceding clauses, where the mitophagy dysfunction-associated disease is a neurodegenerative/neuropsychiatric disease that is Parkinson's disease.
Clause 170. The method according to any of the preceding clauses, where the mitophagy dysfunction-associated disease is a cancer.
Clause 171. The method according to any of the preceding clauses, where the mitophagy dysfunction-associated disease is a cancer selected from the group consisting of acute lymphoblastic leukemia, acute myeloid leukemia, adenoid cystic carcinoma, ampullary carcinoma, bladder cancer, breast cancer, cervical cancer, cholangiocarcinoma, clear cell renal cell carcinoma, colorectal cancer, cutaneous melanoma, ependydoma, esophageal cancer, esophageal squamous cell carcinoma, gastric cancer, gastric carcinoma, gastric adenocarcinoma, glioblastoma, glioma, head and neck squamous cell carcinoma, hepatocellular carcinoma, laryngeal cancer, laryngeal squamous cell carcinoma, leukemia, locally advanced rectal cancer, lung cancer, lymphoma, melanoma, multiple myeloma, non-small cell lung cancer, oral melanoma, oral squamous cell carcinoma, oropharyngeal squamous cell carcinoma, osteosarcoma, ovarian cancer, pancreatic cancer, pancreatic ductal adenocarcinoma, prostate cancer, renal carcinoma, squamous cell carcinoma, tongue squamous cell carcinoma, triple negative breast cancer, and uterine-cervical squamous cell carcinoma.
Clause 172. The method according to any of the preceding clauses, where the mitophagy dysfunction-associated disease is a cancer that is lung cancer.
Clause 173. The method according to any of the preceding clauses, where the SREBP signaling pathway activator is a small molecule SREBP signaling pathway activator.
Clause 174. The method according to any of the preceding clauses, where the SREBP signaling pathway activator is a selective SREBP signaling pathway activator.
Clause 175. The method according to any of the preceding clauses, where the SREBP signaling pathway activator increases expression of a gene encoding SREBP.
Clause 176. The method according to any of the preceding clauses, where the SREBP signaling pathway activator is an NCEH1 inhibitor that blocks enzymatic activity of NCEH1.
Clause 177. The method according to any of the preceding clauses, where the SREBP signaling pathway activator is an NCEH1 inhibitor that reduces expression of a gene encoding NCEH1.
Clause 178. The method according to any of the preceding clauses, where the SREBP signaling pathway activator is an NCEH1 inhibitor that is a siRNA, an antisense oligonucleotide or a ribozyme.
Clause 179. The method according to any of the preceding clauses, where the SREBP signaling pathway activator is an NCEH1 inhibitor that is a small molecule NCEH1 inhibitor.
Clause 180. The method according to any of the preceding clauses, where the SREBP signaling pathway activator is a nonselective NCEH1 inhibitor that is propranolol or R-propranolol.
Clause 181. The method according to any of the preceding clauses, where the SREBP signaling pathway activator is an NCEH1 inhibitor that is a selective NCEH1 inhibitor.
Clause 182. The method according to any of the preceding clauses, where the SREBP signaling pathway activator is a selective NCEH1 inhibitor that is JW480.
Clause 183. The method according to any of the preceding clauses, where the SREBP transcriptional activation is contingent upon nuclear translocation of NCEH1.
Clause 184. The method according to any of the preceding clauses, where the SREBP signaling pathway activator is co-administered with one or more other therapeutic agents. Clause. The method according to any of the preceding clauses, where the SREBP signaling pathway activator is co-administered with one or more other therapeutic agents.
Clause 185. A method to stimulate intracellular mitophagy in a cell nucleus of a subject in need thereof, the method comprising the step of: administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into the cell nucleus or a pharmaceutically acceptable salt thereof sufficient to stimulate the cellular mitophagy in the subject.
Clause 186. The method according to the preceding clause 185, where the neutral cholesterol ester hydrolase 1 (NCEH1) is bound to the SREBP N-terminal transactivation domain.
Clause 187. The method according to any of the preceding clauses 185-186, where the administered SREBP signaling pathway activator induces nuclear translocation of SREBP N-terminal transactivation domain linked to the SREBP C-terminal regulatory domain.
Clause 188. The method according to any of the preceding clauses 185-187, where the administered SREBP signaling pathway activator induces nuclear translocation of intact, full length SREBP.
Clause 189. The method according to any of the preceding clauses 185-188, where the SREBP is an isoform selected from the group consisting of SREBP1a, SREBP1c and SREBP2.
Clause 190. The method according to any of the preceding clauses 185-189, where the SREBP signaling pathway activator that induces nuclear translocation of SREBP N-terminal transactivation domain and SREBP C-terminal regulatory domain into the cell nucleus is a neutral cholesterol ester hydrolase 1 (NCEH1) inhibitor.
Clause 191. A method to stimulate intracellular lipid biosynthesis in a cell nucleus of a subject in need thereof, the method comprising the step of: administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into the cell nucleus or a pharmaceutically acceptable salt thereof sufficient to stimulate the intracellular lipid synthesis in the subject.
Clause 192. The method according to the preceding clause 191, where the neutral cholesterol ester hydrolase 1 (NCEH1) is bound to the SREBP N-terminal transactivation domain.
Clause 193. The method according to any of the preceding clauses 191-192, where the administered SREBP signaling pathway activator induces nuclear translocation of SREBP N-terminal transactivation domain linked to the SREBP C-terminal regulatory domain.
Clause 194. The method according to any of the preceding clauses 191-193, where the administered SREBP signaling pathway activator induces nuclear translocation of intact, full length SREBP.
Clause 195. The method according to any of the preceding clauses 191-194, where the SREBP is an isoform selected from the group consisting of SREBP1a, SREBP1c and SREBP2.
Clause 196. The method according to any of the preceding clauses 191-195, where the SREBP signaling pathway activator that induces SREBP C-terminal regulatory domain translocation into the cell nucleus is a neutral cholesterol ester hydrolase 1 (NCEH1) inhibitor.
Clause 197. A method to treat an intracellular mitophagy dysfunction-associated disease in a subject in need thereof, the method comprising the step of: administering to the subject an effective amount of a sterol regulatory element-binding protein (SREBP) signaling pathway activator that induces nuclear translocation of SREBP N-terminal transactivation domain and neutral cholesterol ester hydrolase 1 (NCEH1) into a cell nucleus or a pharmaceutically acceptable salt thereof.
Clause 198. The method according to the preceding clause 197, where the neutral cholesterol ester hydrolase 1 (NCEH1) is bound to the SREBP N-terminal transactivation domain.
Clause 199. The method according to any of the preceding clauses 197-198, where the administered SREBP signaling pathway activator induces nuclear translocation of SREBP N-terminal transactivation domain linked to the SREBP C-terminal regulatory domain.
Clause 200. The method according to any of the preceding clauses 197-199, where the administered SREBP signaling pathway activator induces nuclear translocation of intact, full length SREBP.
Clause 201. The method according to any of the preceding clauses 197-200, where the SREBP is an isoform selected from the group consisting of SREBP1a, SREBP1c and SREBP2.
Clause 202. The method according to any of the preceding clauses 197-201, where the SREBP signaling pathway activator that induces nuclear translocation of SREBP N-terminal transactivation domain and SREBP C-terminal regulatory domain in the cell nucleus is a neutral cholesterol ester hydrolase 1 (NCEH1) inhibitor.
Clause 203. A method of enhancing mitophagy in a cell, said method comprising the step of contacting said cell with an activator of the sterol regulatory element-binding protein (SREBP) pathway.
Clause 204. The method of any one of clauses 1-203 wherein the activator of the SREBP pathway is a compound selected from the group consisting of propranolol, R-propranolol, papuamide, U18666A and JW480.
Clause 205. The method of Clause 204 wherein the activator of the SREBP pathway is R-propranolol.
Clause 206. The method of Clause 204 wherein the activator of the SREBP pathway is propranolol.
Clause 207. The method of Clause 204 wherein the activator of the SREBP pathway is JW480.
Clause 208. The method of any one of Clauses 203-207 wherein said cell is contacted in vivo by administering the activator of the SREBP pathway to a subject.
Clause 209. The method of Clause 208 wherein the subject has a mitophagy dysfunction-associated disease.
Clause 210. A method to stimulate intracellular lipid biosynthesis in a cell of a subject in need thereof, the method comprising the step of: administering to the subject an effective amount of a composition comprising an activator of the sterol regulatory element-binding protein (SREBP) pathway sufficient to stimulate the intracellular lipid synthesis in the subject.
Clause 211. The method of Clause 210 wherein the activator of the SREBP pathway is a compound selected from the group consisting of propranolol, R-propranolol, papuamide, U18666A and JW480.
Clause 212. The method of Clause 210 wherein the activator of the SREBP pathway is propranolol.
Clause 213. The method of Clause 210 wherein the activator of the SREBP pathway is R-propranolol.
Clause 214. The method of Clause 210 wherein the activator of the SREBP pathway is JW480.
Clause 215. A method to identify a biomarker for cellular mitophagy in a target tissue of a mammal, the method comprising the steps of:
(a) administering a sterol regulatory element-binding protein (SREBP) signaling pathway activator that induces SREBP C-terminal regulatory domain translocation into the cell nucleus or a pharmaceutically acceptable salt thereof, to the mammal;
(b) collected endothelial cells from a blood sample of the mammal;
(c) analyzing the collected mammalian endothelial cells for cellular mitophagy;
(d) collecting cells from the target tissue of the mammal;
(e) analyzing the collected mammalian target tissue cells for cellular mitophagy; and
(f) determining a correlation between cellular mitophagy in the mammalian endothelial cells and cellular mitophagy in the mammalian target tissue cells.
Clause 216. A method of identifying a candidate sterol regulatory element-binding protein (SREBP) signaling pathway activator, the method comprising the steps of:
(a) contacting a cell with the candidate SREBP signaling pathway activator; and,
(b) analyzing the contacted cell for mitophagy and for presence of SREBP C-terminal regulatory domain or for presence of SREBP N-terminal transactivation domain and neutral cholesterol ester hydrolase 1 (NCEH1) translocation in the cell nucleus.
Clause 217. A method to stimulate intracellular mitophagy in a cell of a subject in need thereof, the method comprising the step of: administering to the subject an effective amount of propranolol or a pharmaceutically acceptable salt thereof sufficient to stimulate the cellular mitophagy in the subject.
Clause 218. A method to stimulate intracellular lipid biosynthesis in a cell of a subject in need thereof, the method comprising the step of: administering to the subject an effective amount of R-propranolol, JW480 or a pharmaceutically acceptable salt thereof sufficient to stimulate the intracellular lipid synthesis in the subject.
Clause 219. A method to treat an intracellular mitophagy dysfunction-associated disease in a subject in need thereof, the method comprising the step of: administering to the subject an effective amount of propranolol or a pharmaceutically acceptable salt thereof.
Clause 220. A method of identifying a biomarker for cellular mitophagy in a target tissue of a mammal, by: (a) administering a sterol regulatory element-binding protein (SREBP) signaling pathway activator that induces SREBP C-terminal regulatory domain translocation into the cell nucleus or a pharmaceutically acceptable salt thereof, to the mammal; (b) collected endothelial cells from a blood sample of the mammal; (c) analyzing the collected mammalian endothelial cells for cellular mitophagy; (d) collecting cells from the target tissue of the mammal; (e) analyzing the collected mammalian target tissue cells for cellular mitophagy; and (f) determining a correlation between cellular mitophagy in the mammalian endothelial cells and cellular mitophagy in the mammalian target tissue cells.

### EXAMPLES

### Materials and Methods

Western blot. Cell lysates were resolved on a SDS PAGE pre-cast gel (Invitrogen) and resolved proteins were transferred on to a PVDF membrane for further detection of proteins using specific antibodies. Antibodies for SREBP1 were purchased from Santa Cruz Biotechnology (Cat #sc-365513) and Novus biological (Cat #NB100-2215). SCD antibody was purchased from Santa Cruz Biotechnology (Cat #sc-58420). Tom20 and LAMP1 antibodies were purchased from Cell Signaling (Cat #42406S and 15665S, respectively).

Immunofluorescence Microscopy. Cells were fixed with 4% Formaldehyde (Electron Microscopy Sciences, #C993M24) and permeabilized in the presence of 0.1% Triton X-100 (Sigma). Full-length SREBP1 was stained using mouse monoclonal antibody from Santa Cruz Biotechnology (Cat #sc-365513). Whereas nuclear SREBP1 was detected using antibody from Novus biological (Cat #NB100-2215). Mitophagy was confirmed by staining mitochondrial Tom20 and lysosomal marker LAMP1. Tom20 and LAMP1 antibodies were purchased from Cell Signaling (Cat #42406S and 15665S, respectively). Images were captured on an inverted fluorescent microscope (Leica).

Transmission Electron Microscopy. Cells were exposed to vehicle or 10 uM propranolol or 10 uM R-propranolol or 0.1 uM JW480 for 12 hours and fixed with fixative supplied by in house Electron Microscopy Core at Indiana University. Thin sections from these fixed cell blocks were visualized on Tecnai Spirit (ThermoFisher) TEM Microscope. Images captured were analyzed.

Immunoprecipitation Assay. In determining endogenous SREBP1-NCEH1 complex formation, cultured endothelial cells were treated with IP lysis buffer (Pierce Cat #87787). SREBP1 complex was captured using SREBP1 antibody from Santa Cruz Biotechnology (Cat #sc-365513) and resolved on a SDS PAGE. Complex formation with NCEH1 was detected by blotting with NCEH1 antibodies (Sigma Cat #HPA026888). To map the domain of SREBP1 that binds to NCEH1, HA tagged full-length SREBP1 or C-terminal regulatory domain alone were co-expressed along with Flag tagged NCEH1 in HEK293 cells. SREBP proteins were captured on HA antibodies (Cell Signaling) and blotted with Flag antibodies (Sigma).

NCEH1 knockdown experiments. NCEH1 was depleted using siRNA pool from Dharmacon or Lentiviral shRNA from Sigma. NCEH1 depletion was confirmed on a Western blot or by immunofluorescence assay.

Rat MCT model of PAH. SD rats were given one dose of MCT injection to induce pulmonary hypertension. Three weeks of post MCT injections, these rats were given 5 mg/kg or 10 mg/kg propranolol for one week. Rats were anesthetized and RVSP recorded.

### EXAMPLES

### Example 1—Propranolol Promoted Mitophagy in Arterial Hypertension (PAH) Derived Pulmonary Arterial Endothelial Cells (PAECs)

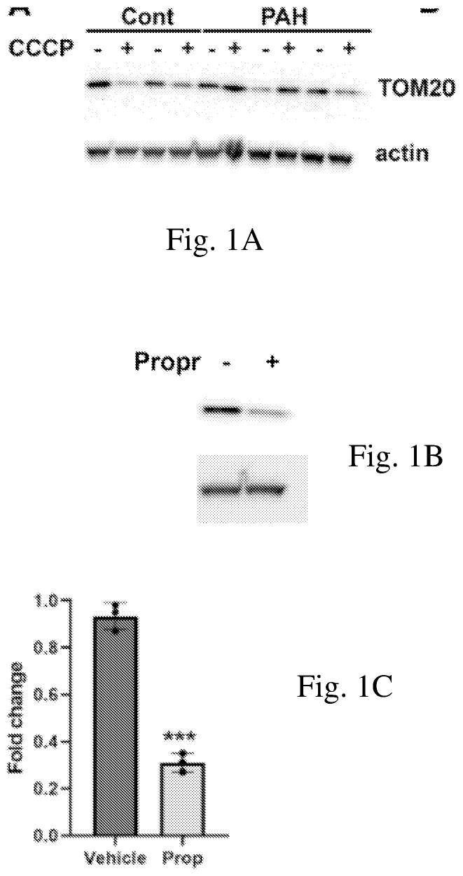

“Donor” (non-PAH control)- and PAH-derived PAEC were treated with carbonyl cyanide m-chlorophenylhydrazone (CCCP), an ionophore commonly used to induce mitophagy. While CCCP induced mitophagy in donor-derived PAEC (low TOM20), consistent effects in PAH-derived PAEC (FIG. 1A; FIG. 1C quantitates data from FIG. 1A) were not observed. These data established that patient-derived cells are recalcitrant to mitophagy induction and indicated that any drug that can restore mitophagy may potentially reverse the pathological state of these cells.

PAH patient-derived PAEC was exposed to propranolol and induction of mitophagy was assessed via two methods. First, degradation of a mitochondrial marker (TOM20) was evaluated as a bio-chemical marker of mitophagy induction. Propranolol-exposed PAEC showed reduced TOM20 levels, consistent with increased mitophagy (FIG. 1B). Second, propranolol treated PAEC were co-stained with TOM20 and a lysosomal marker (LAMP1). The results showed reduced TOM20/Lamp1 co-staining with propranolol, providing microscopic immunofluorescence evidence of mitophagy induction, which confirmed evidence of autophagy of mitochondria. In contrast to CCCP, propranolol effectively reduced TOM20 in PAH-derived PAEC (FIG. 1B). The data indicated: 1) pharmacological induction of mitophagy is impaired in PAH-derived PAEC; and 2) propranolol enables induction of mitophagy in PAH-derived PAEC. Referring to FIGS. 1A-1C, it is demonstrated that propranolol promotes mitophagy in PAECs.

### Example 2-Propranolol Restored Mitophagy in Control and Patient Cells

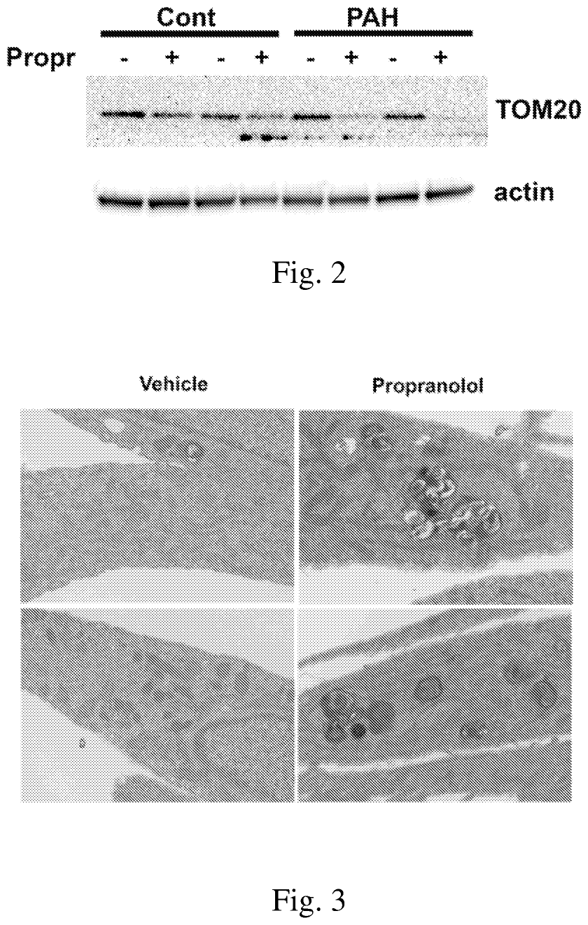

Although carbonyl cyanide m-chlorophenyl hydrazine (CCCP) promotes mitophagy in ECs from normal control subjects, it failed to promote mitophagy in patient-derived cells. Normal cells and PAH patient cells were treated with propranolol and mitophagy was assessed. Propranolol consistently decreased Tom20 levels in PAH patient cells much more robustly than in normal cells. Referring to FIG. 2, it is shown that propranolol promotes mitophagy in patient cells. Normal control ECs or patient-derived ECs were treated with propranolol and Tom20 levels were assessed. These results evidence that propranolol is selectively promoting mitophagy in patient cells.

### Example 3-Mitophagy by Propranolol-Electron Microscopy Evidence

Although Tom20 western blot and immunofluorescence data indicated that propranolol promotes mitophagy in ECs, transmission electron microscopy (TEM) is a commonly accepted “gold standard” method for mitophagy. To obtain TEM evidence for mitophagy induction by propranolol, cells exposed to propranolol were processed for TEM and the imaged captured on a TEM. The results established that propranolol promotes bilipid membrane formation around mitochondria more frequently than vehicle treated cells.

### Example 4-Propranolol Reversed PAH Disease in Rats

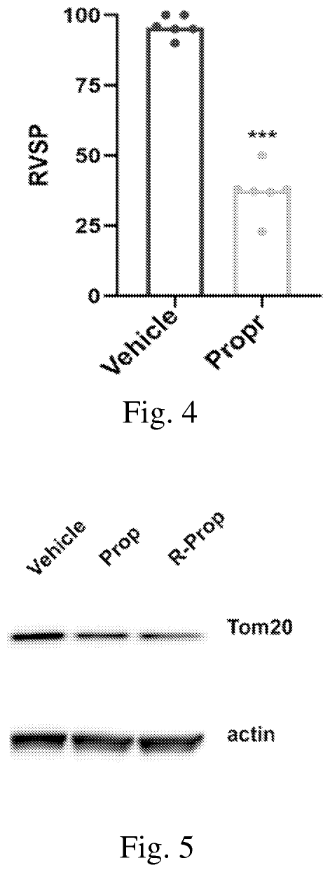

Rats were injected with MCT to develop PAH in a well-established 4 week rodent model of the disease. Rats were treated with vehicle or propranolol (10 mg/kg, intraperitoneal or IP) after 2 weeks of allowing MCT to develop disease. Propranolol significantly reduced right ventricle systolic pressures (RVSP), a well-recognized measure of PAH in rats, and negated MCT-induced arterial wall thickness (FIG. 4) arising from the PAH-related obliterative vascular remodeling. Referring to FIG. 4, data from RVSP measurements of rats exposed to MCT and treated with vehicle or propranolol for the last 2 of a 4-week PAH show that propranolol attenuates MCT-induced PAH in rats. Rat lung sections were stained with H&E, and histology analysis showed vessels were much less remodeled and vessel thickness is attenuated after exposure to propranolol, further supporting propranolol's ability to attenuate PAH lung vascular remodeling and disease pathogenesis.

### Example 5-Propranolol Activated SREBP1 Pathway and Lipid Synthesis

SREBP1 is a key transcription factor that promotes lipid synthesis upon activation. Normally, SREBP1 protein is retained in the endoplasmic reticulum by high levels of cholesterol. When the cholesterol levels drop, SREBP1 protein gets translocated to the Golgi where it is cleaved, and the transcriptional activation portion of this protein gets translocated to the nucleus. Subsequently, SREBP1 activates a transcription program of genes involved in lipid synthesis. RNAseq data was generated to study mechanisms of propranolol-mediated PAEC mitophagy induction. Pathway analysis revealed significant induction of the sterol regulatory element-binding transcription factor (SREBP) pathway, an established regulator of lipid metabolism. ECs were treated with propranolol and the total RNA isolated from the cells were subjected to high through-put RNA sequencing. Analysis of the RNA-seq data revealed that propranolol activated transcriptional targets, as well as transcript levels of SREBP1. The pathway analysis established that propranolol promotes activation of SREBP1 pathway. Published genome-wide association studies (GWASs) in patients have revealed that SREBP is a risk locus for sporadic Parkinson disease (PD). The data in the context of these published observations for SREBP as a gene relevant in PD indicate that propranolol activates SREBP1 and lipid synthesis in promoting mitophagy and, because of its effects on mitophagy, reverses PD as one of many pathological conditions.

### Example 6-R-Propranolol Promoted Mitophagy

Propranolol is a first generation non-selective beta-blocker and robustly inhibits beta-adrenergic receptors on cell membranes. R-propranolol, its enantiomer, has 100-fold or less affinity for beta-adrenergic receptors and thus, its use is considered a pharmacological strategy to show beta-adrenergic receptor-independent mechanisms. R-propranolol was tested to determine whether it activates mitophagy. ECs were exposed propranolol or R-propranolol and Tom20 levels were assessed. The results showed that similar to propranolol, R-propranolol also decreased Tom20 levels indicating that propranolol promotes mitophagy in part, via, a beta-receptor independent manner (FIG. 5).

### Example 7-SREBP1 Activation Promoted Mitophagy

ECs were exposed to propranolol or R-propranolol or SREBP1 activator U18666A and mitophagy was assessed by IF (immunofluorescence microscopy). All three drugs induce mitophagy based on the immunofluorescence data. The results revealed that similar to propranolol, SREBP1 activation also promoted mitophagy. These results demonstrated that SREBP1 activation is a powerful strategy to promote mitophagy that impacts many pathological conditions that require activation of mitophagy.

### Example 8-SREBP1 Inhibition Partially Blocked Mitophagy

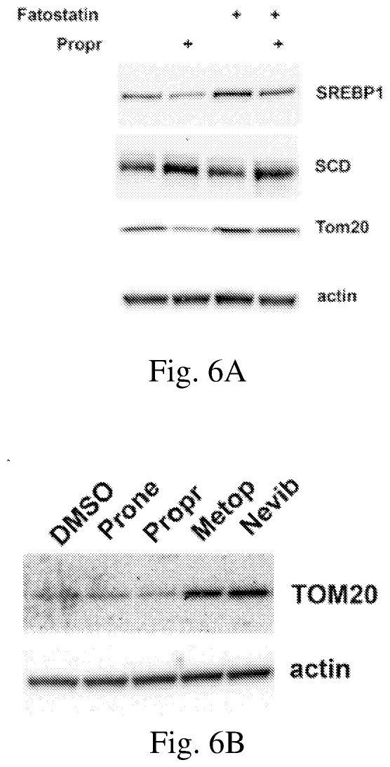

As lipids are degraded in the mitochondria, their synthesis and mitophagy can be regulated nonreciprocally. To confirm RNAseq data, PAEC was treated with propranolol with an SREBP1 inhibitor, fatostatin, and both were assessed with an SREBP1 target, stearoyl-CoA desaturase (SCD), and TOM20. Data showed that propranolol-induced mitophagy was partially reversed by fatostatin (FIG. 6A). These results indicate that propranolol targets both a classical SREBP1 pathway and a non-classical pathway.

### Example 9-Propranolol Targeted NCEH1 in Activation of SREBP1

The Similarity Ensemble Approach (SEA) is an online tool that relates proteins based on the set-wise chemical similarity among their ligands. It rapidly searches large compound databases to build cross-target similarity maps. Using this tool, the proteins that are potential targets of propranolol, pronetalol, metoprolol and nebivolol were analyzed (FIG. 6B). Although metoprolol and nebivolol are used as selective beta blockers but are only used in treating cardiovascular disorders generally, these drugs failed to dock to neutral cholesterol ester hydrolase 1 (NCEH1). These two drugs failed to promote mitophagy as well. However, propranolol, a non-selective beta blocker robustly induced mitophagy and docks to NCEH1 with high degree of confidence. These results from clinical settings and in silico studies indicated that propranolol targets NECH1 to activate mitophagy in disease conditions and presents NCEH1 blockage as a strategy to activate mitophagy and reverse many pathological conditions.

### Example 10-A Proposed Model of NCEH1-Mediated SREBP1 Activation

NCEH1 is cholesterol ester hydrolase and a membrane bound protein. A model is proposed wherein membrane/lipid bound NCEH1 binds to the regulatory domain of SREBP1 and retains it in the cytosol. Propranolol competes out lipid binding of NCEH1 and releases an NCEH1-SREBP1 complex to translocate into the nucleus.

### Example 11-NCEH1 Inhibition Activated SREBP1 and Promoted Mitophagy

ECs were treated with vehicle or propranolol or R-propranolol or 100 times lesser concentration of JW480, an inhibitor of NCEH1, and blotted to assess SREBP1 activation and mitophagy. The blot showed that similar to propranolol or R-propranolol, 0.1 uM NCEH1 was sufficient to activate SREBP1 target SCD and reduced the levels of Tom20, an indication of mitophagy (FIG. 7B).

### Example 12-NCEH1 Inhibition Promoted Mitophagy

ECs were exposed to vehicle or propranolol or R-propranolol or JW480 and mitophagy induction was evaluated by immunofluorescence assay. Fixed cells were stained with Tom20 and Lamp1 and the fluorescence images were captured on a fluorescence microscope. The results showed that propranolol or R-propranolol or JW480 treated cells, Tom20 was mobilized to lysosome as evident from co-localization and specle formation with Lamp1 staining. These results indicated that NCEH1 inhibition of SREBP1 activation stimulates induction of mitophagy.

### Example 13-NCEH1 Inhibition Promoted Mitophagy

TEM evidence. ECs were exposed to vehicle or propranolol or R-propranolol or JW480 and mitophagy induction was evaluated by TEM for mitophagy. ECs exposed to these drugs were fixed and the images were captured on a TEM. The results showed that propranolol or R-propranolol or JW480 treatment promoted mitophagy because the treatment caused bilipid membrane formation surrounding mitochondria as well as fusion with lysosome.

### Example 14-NCEH1 Inhibition Promoted Nuclear Translocation of SREBP1

ECs were exposed to vehicle or propranolol or R-propranolol or JW480, and SREBP1 and nuclear accumulation was assessed by IF. The results showed that similar to propranolol, NCEH1 selective inhibitor JW480 promoted nuclear translocation of SREBP1.

### Example 15-NCEH1 Depletion Promoted Nuclear Translocation of SREBP1

Although pharmacological inhibition of NCEH1 promoted nuclear accumulation of SREBP1, genetic evidence further supports these observations. To this end, ECs were transfected with control or siNCEH1 siRNA and localization of SREBP1 or NCEH1 was captured on IF microscope. IF images revealed that reduction in NCEH1 protein correlated with increased in nuclear accumulation of SREBP1.

### Example 16-NCEH1 was Bound to SREBP1

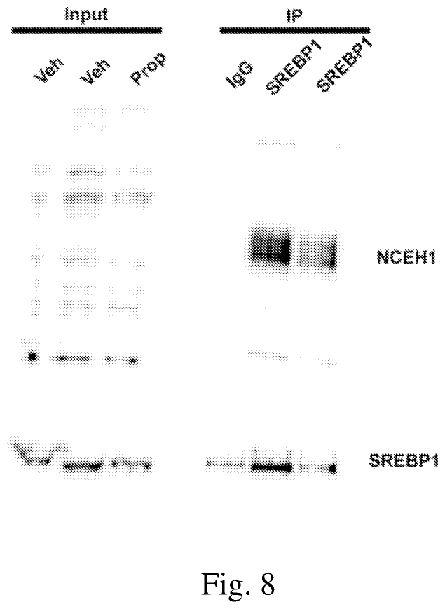

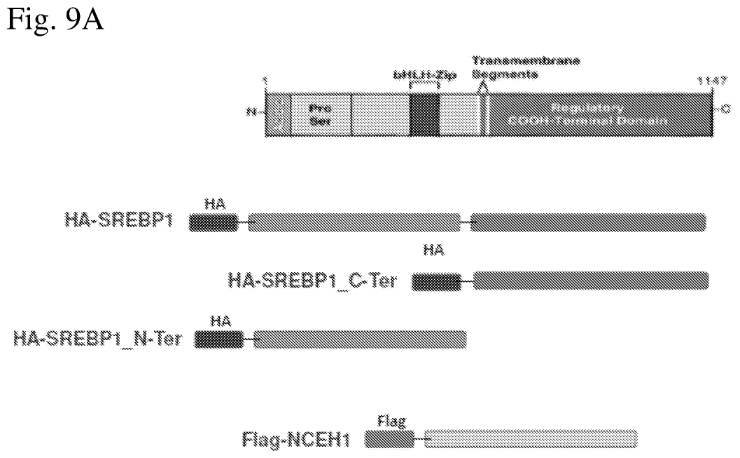

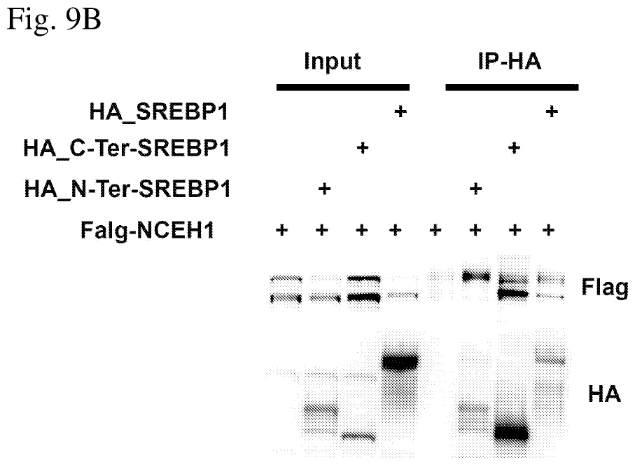

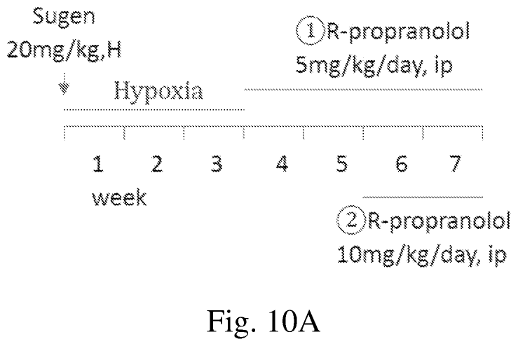

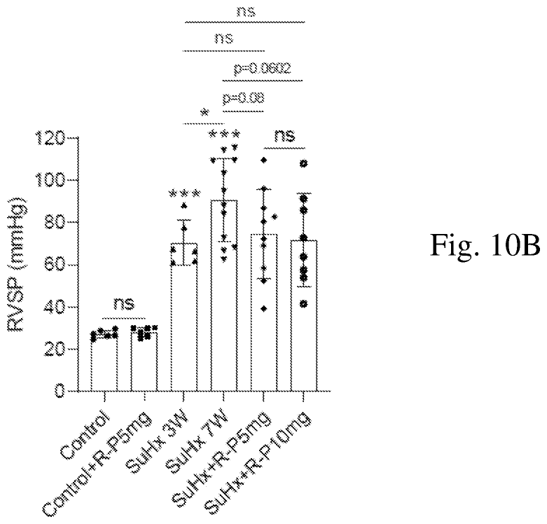

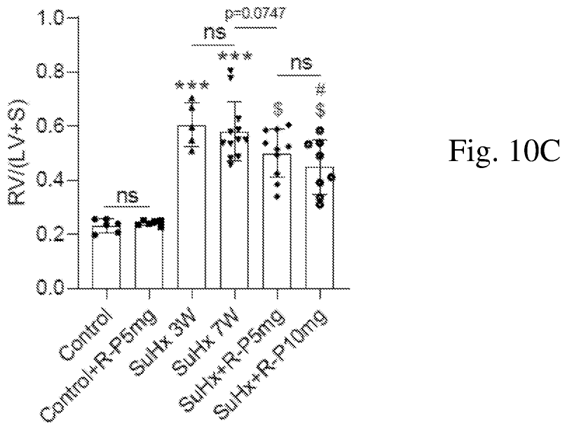

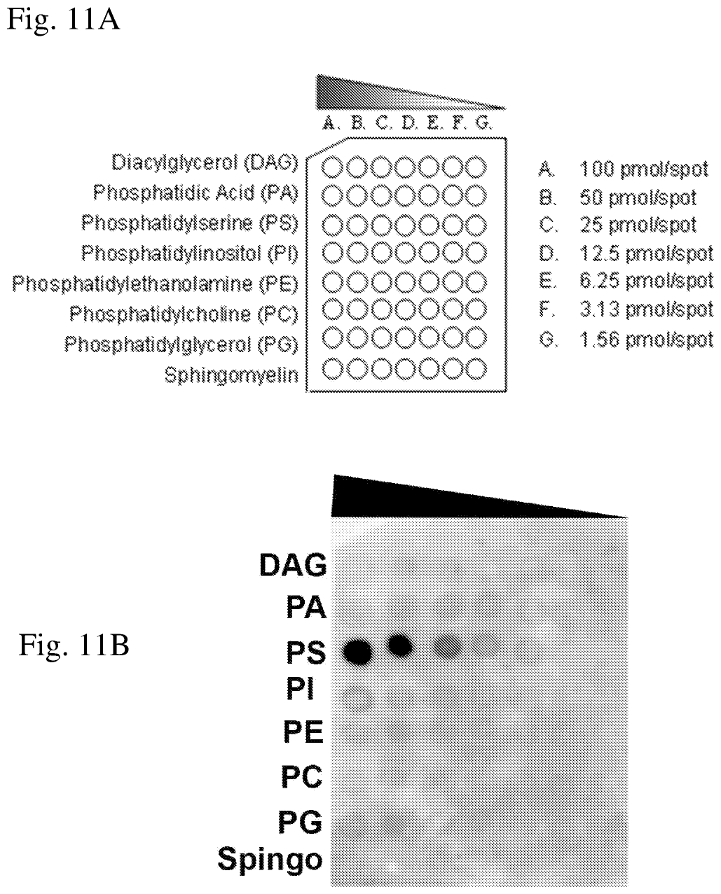

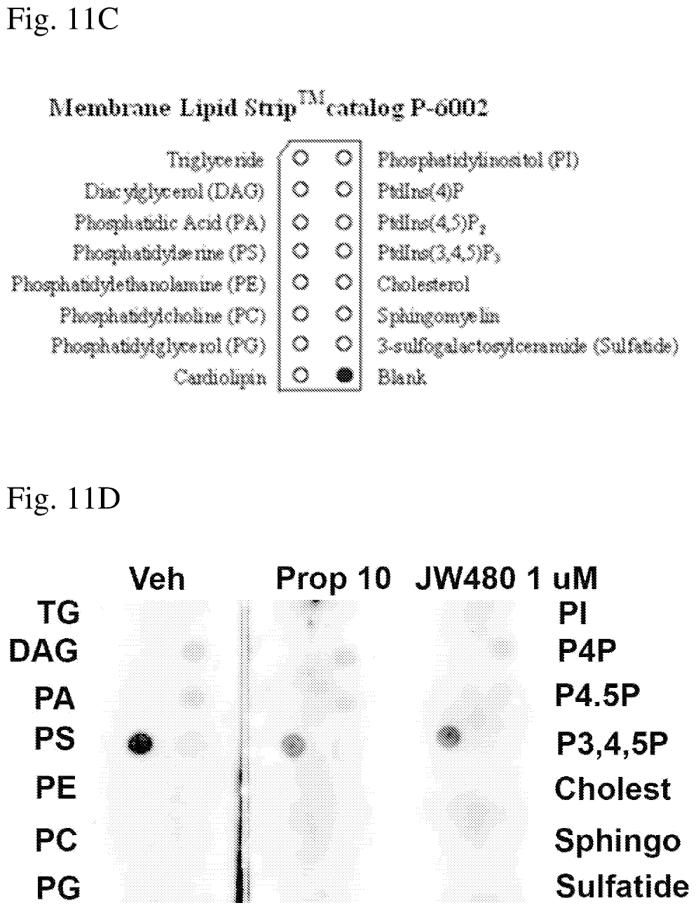

Endogenous SREBP1 was captured on anti-SREBP1 antibody beads and blotted with NCEH1 antibody from ECs treated with vehicle or propranolol. The results (FIG. 8) showed that in vehicle treated cells, NCEH1 coprecipitated with SREBP1. In the case of propranolol treated cells, there was less full length SREBP1 immunoprecipitated and accordingly reduced NCEH1 complexed with full length SREBP1.

These results revealed that propranolol promotes classical pathway cleavage of SREBP1 in addition to a non-classical NCEH1 pathway.

### Example 17-NCEH1 Interacted with SREBP1 at the Transactivation Domain

HEK293 cells were transfected with SREBP1 C-terminal regulatory domain mutant or SREBP1 full length proteins that are HA tagged along with Flag tagged NCEH1. SREBP1 proteins were captured on HA antibody beads and blotted with Flag antibodies to detect NCEH1 interaction with SREBP1 proteins. These data revealed that NCEH1 interacted with transactivation domain of SREBP1.

### Example 18

R-propranolol attenuated rodent PAH. Rats were administered a one-time sugen (20 mg/kg, IP) injection and exposed to 3 weeks of hypoxia (10% FiO2) and then placed in normoxia (21% FiO2) for 4 weeks. This is a well-established second mode of PAH, in addition to data previously showed in FIG. 4 in a monocrotaline rat model. In the last 4 week normoxic period, rats were administered either r-propranolol (5 mg/kg/day, IP) for the last 4 weeks or r-propranolol (10 mg/kg/day, IP) for the last 2 weeks or vehicle for the last 4 weeks. Rats with vehicle exposure developed severe PAH at the end of the protocol as confirmed by right ventricular systolic pressures (RVSP) and surrogate measures of RV hypertrophy (RV/(LV+S)). R-propranolol exposure attenuated both RVSP and RV hypertrophy at the higher dose (10 mg/kg/day) and trended toward improvement with the lower dose (5 mg/kg/day).

Other variations or embodiments will be apparent to a person of ordinary skill in the art from the above-description. Thus, the foregoing embodiments are not to be construed as limiting the scope of the claimed invention. All references disclosed are expressly incorporated by reference in in their entirety.

### Example 19 NCEH1 Inhibitor Improves CV Injury in Two Mouse Models

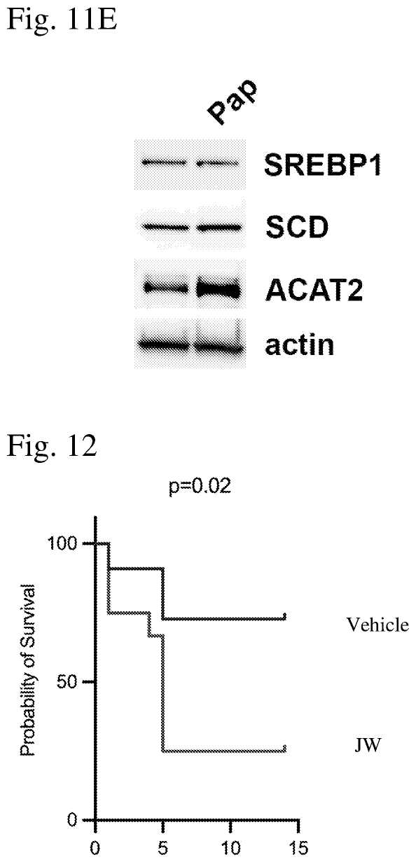

Ligation of the left coronary artery is a well-accepted mouse model of myocardial infarction (MI), associated with high mortality (>70% in lwk). As propranolol was originally developed to treat hypertension and angina pectoris, we hypothesized that JW480 may protect against an MI. Mice were treated with vehicle or 20 mg/kg JW480 (IP, n=6 males & 6 females) for five days and subjected to an MI. The results showed significantly improved survival of JW480-preconditioned mice compared to control mice (FIG. 12). Transmission electron microscopy (TEM) data from JW480-treated mice show increased intracellular lipid droplets, a hallmark for SREBP1 activation. These data are consistent with a prior report using SREBP1 knockout mice that suggest SREBP1 protects mice from arrhythmias.

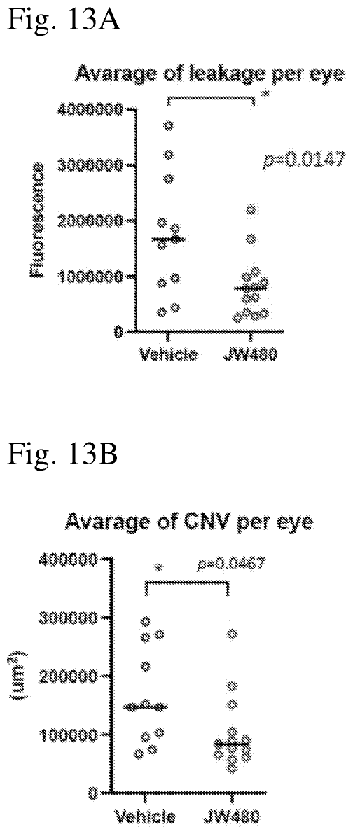

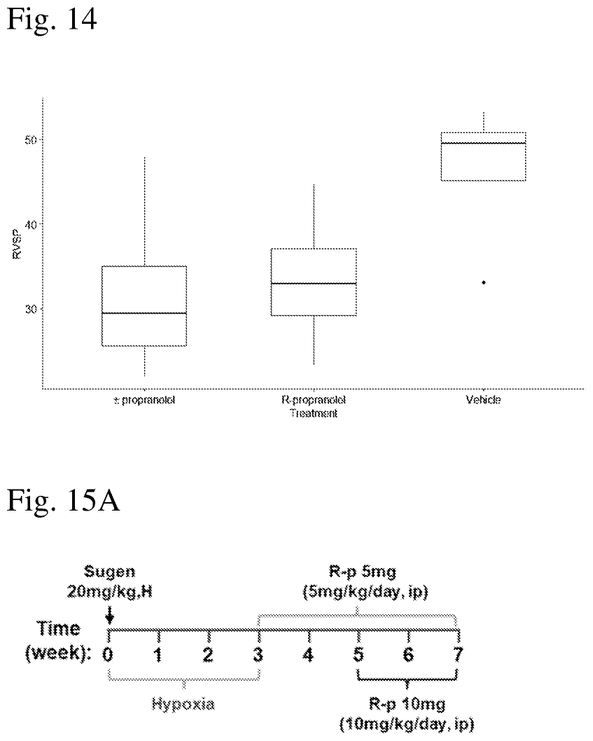

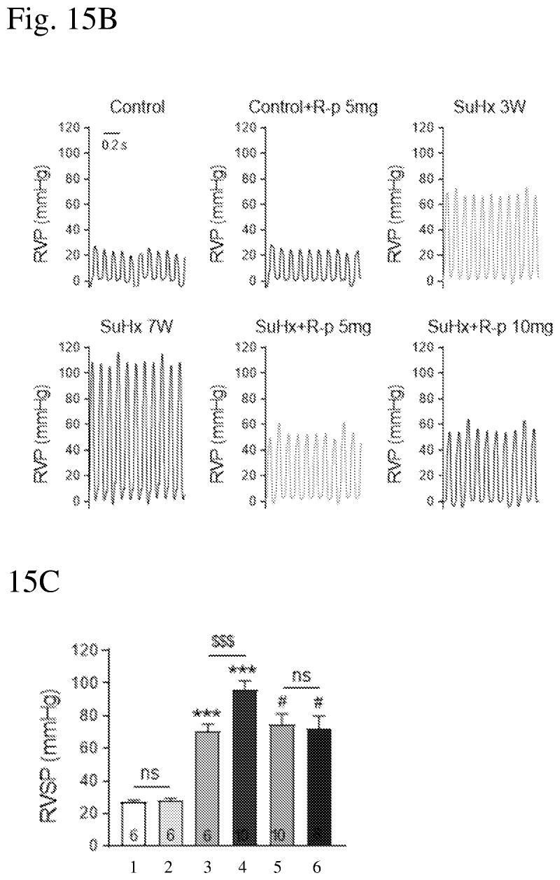

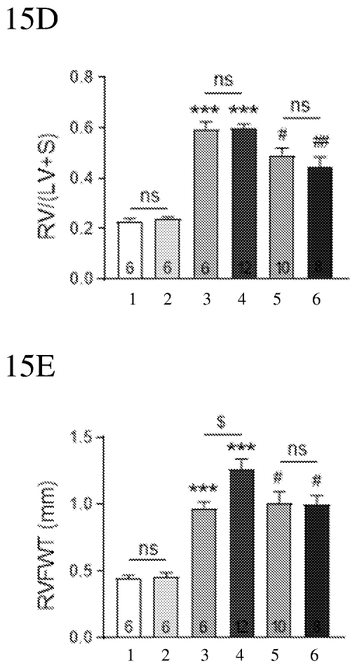

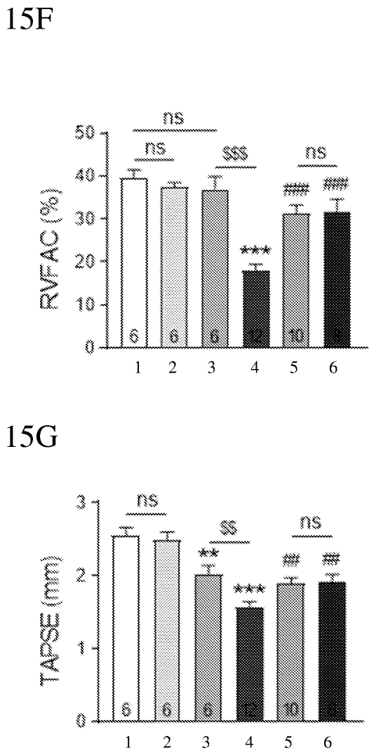

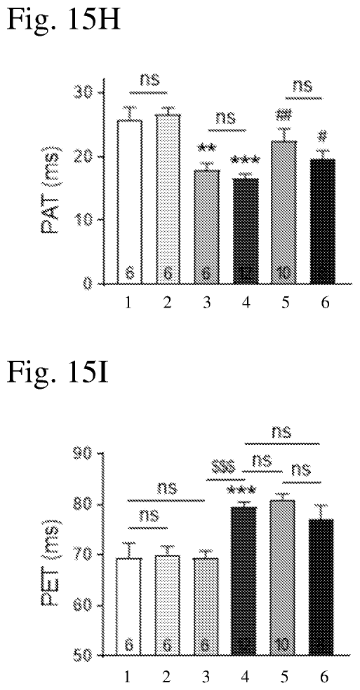

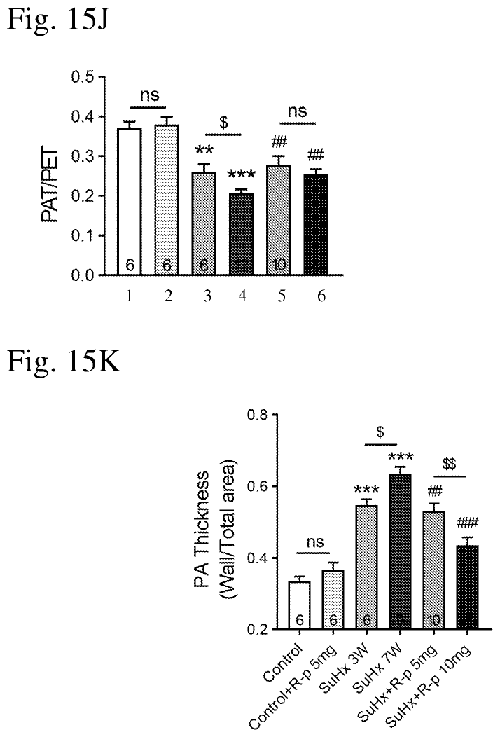

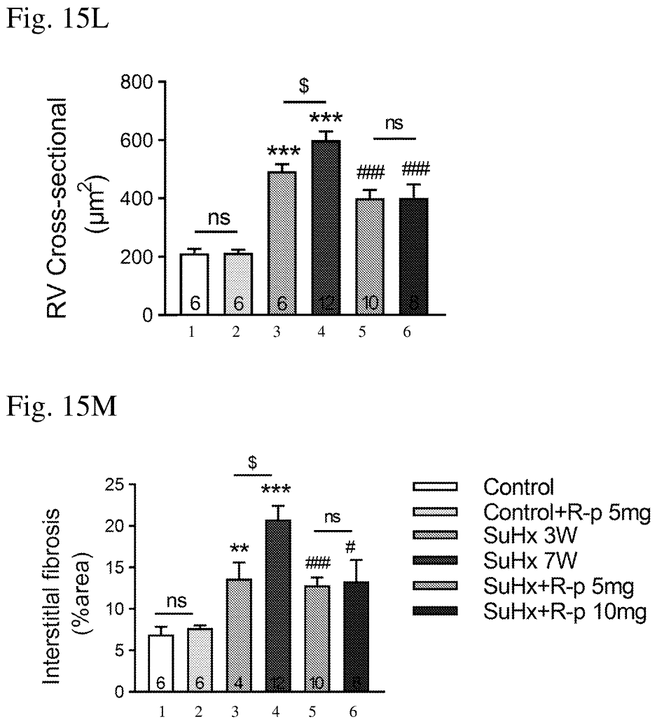

The eye offers an opportune model to study vascular pathology. The laser-induced choroidal neovascularization (CNV)) mouse model is used to study pathologic exudative angiogenesis as a major contributor to age-related macular degeneration (AMD). To test the protective effects of NCEH1 blockade, CNV was induced in two groups of mice that are exposed to either vehicle or 20 mg/kg of JW480 for seven days (n=12 each). JW480 treatment significantly reduced retinal vessel leakage and inhibited pathologic CNV (FIGS. 13A & 13B). Cumulatively, these mice studies provide evidence for the therapeutic use of JW480 in pathological CV conditions.

### Example 20

Using a mouse model fed a high-fat diet (Research Diets, New Brunswick, NJ) for 16-20 weeks, AKR/J mice (Jackson Laboratories, Bar Harbor, ME) develop obesity, insulin resistance, heart failure with preserved ejection fraction, and pulmonary hypertension. (Meng, et al., 2017; Arwood, et al., 2019). We treated these HF-PH mice with either, racemic propranolol (non-selective beta-adrenergic receptor antagonist), R-propranolol (little to no beta-adrenergic receptor activity), or vehicle for 4 weeks using an implanted mini osmotic pump. We observed significantly decreased right ventricular systolic pressure (indicator of pulmonary hypertension) in both propranolol and R-propranolol (FIGS. 1A-1C) compared to vehicle (P=0.05). These results suggest that the improvement in PH is not due to the beta-adrenergic antagonist properties of propranolol.

1. A method of enhancing mitophagy in a cell, said method comprising the step of contacting said cell with an activator of the sterol regulatory element-binding protein (SREBP) pathway.

2. The method of claim 1 wherein the activator of the SREBP pathway is a compound selected from the group consisting of propranolol, R-propranolol, papuamide, U18666A and JW480.

3-5. (canceled)

6. The method of claim 2 wherein said cell is contacted in vivo by administering the activator of the SREBP pathway to a subject having a mitophagy dysfunction-associated disease.

7. (canceled)

8. The method of claim 6 wherein the mitophagy dysfunction-associated disease is selected from the group consisting of mitochondrial disease, lung/respiratory disease, cardiovascular disease, liver disease, renal disease, neurodegenerative/neuropsychiatric disease and cancer.

9. The method of claim 6 wherein the mitophagy dysfunction-associated disease is a lung/respiratory disease selected from the group consisting of bronchopulmonary dysplasia, chronic obstructive pulmonary disease, lung cancer, asthma, cystic fibrosis, pulmonary arterial hypertension, inflammatory lung disease, pleural cavity disease, pulmonary vascular disease, pneumonia, pulmonary embolism, idiopathic pulmonary fibrosis, sarcoidosis, mixed connective tissue disease, polymyositis, dermatomyositis, and systemic lupus erythematosus.

10. The method of claim 6 wherein the lung/respiratory disease is pulmonary hypertension or pulmonary arterial hypertension.

11. The method of claim 6 wherein the mitophagy dysfunction-associated disease is a cardiovascular disease selected from the group consisting of acute cardiac ischemic events, acute myocardial infarction, angina, arrhythmia, atherosclerosis, and chronic heart failure.

12. A method of increasing nuclear translocation of SREBP C-terminal regulatory domain into a cell nucleus, said method comprising the steps of contacting said cell with an activator of the sterol regulatory element-binding protein (SREBP) pathway.

13. The method of claim 12 wherein the activator of the SREBP pathway is a compound selected from the group consisting of propranolol, R-propranolol, papuamide, U18666A and JW480.

14-17. (canceled)

18. The composition of claim 13 comprising two or more compounds selected from the group consisting of propranolol, U18666A and JW480.

19. A method of treating mitophagy dysfunction or stimulate intracellular lipid biosynthesis in a cell of a subject in need thereof, said method comprising the step of contacting a cell exhibiting mitophagy dysfunction with a composition comprising an activator of the sterol regulatory element-binding protein (SREBP) pathway in an amount sufficient to increase nuclear translocation of SREBP C-terminal regulatory domain into the nucleus of said cell.

20. The method of claim 19 wherein the activator of the SREBP pathway is a compound selected from the group consisting of propranolol, R-propranolol, papuamide, U18666A and JW480.

21. The method of claim 20 wherein the activator of the SREBP pathway is propranolol.

22. The method of claim 20 wherein the activator of the SREBP pathway is R-propranolol.

23. The method of claim 20 wherein the activator of the SREBP pathway is JW480.

24. The method of claim 1 wherein said cell is a cell of a subject in need of increased intracellular mitophagy, the method comprising the step of: administering to the subject an effective amount of a an activator of the sterol regulatory element-binding protein (SREBP) pathway selected from the group consisting of R-propranolol, U18666A, JW480 and pharmaceutically acceptable salts thereof, sufficient to stimulate the cellular mitophagy in the subject.

25. (canceled)

26. The method of claim 24 wherein the activator of the SREBP pathway is a compound selected from the group consisting of propranolol, R-propranolol, papuamide, U18666A and JW480.

27. The method of claim 26 wherein the activator of the SREBP pathway is propranolol.

28. The method of claim 26 wherein the activator of the SREBP pathway is R-propranolol.

29. The method of claim 26 wherein the activator of the SREBP pathway is JW480.

30. (canceled)
